(19) United States
(12) Patent Application Publication
(10) Pub. No.: US 2016/0317441 A1
(43) Pub. Date: Nov. 3, 2016

(54) BIOMEMBRANE PHASE-CHANGE DROPLETS (PCD), DRUG CARRIER AND USE THEREOF

(71) Applicant: NATIONAL TSING HUA UNIVERSITY, Hsinchu (TW)

(72) Inventors: Chien-Wen CHANG, Hsinchu (TW); Chen-Chan HSIEH, Hsinchu (TW); Chih-Kuang YEH, Hsinchu (TW); Yee-Hsien LIN, Hsinchu (TW); Shih-Tsung KANG, Hsinchu (TW); Pei-Ying CHEN, Hsinchu (TW)

(21) Appl. No.: 14/944,033

(22) Filed: Nov. 17, 2015

(30) Foreign Application Priority Data
Apr. 30, 2015 (TW) 104113844

(51) Int. Cl. A61K 9/127; A61K 31/4375; A61K 49/22

(52) U.S. Cl. CPC A61K 9/127; A61K 49/227; A61K 31/4375

(57) ABSTRACT

The present invention provides a biomembrane phase-change droplet (PCD), including a hydrophobic liquid core; and a phospholipid-containing biomembrane encapsulating the hydrophobic liquid core, wherein the hydrophobic liquid core is vaporized by ultrasonic irradiation. The present disclosure also provides a drug carrier, including a biomembrane phase-change droplet; and a hydrophobic drug embedded on the biomembrane of the biomembrane phase-change droplet, wherein the hydrophobic drug is presented in an amount of 1-10 wt %, based on the weight of the drug carrier. The present invention further provides the use of aforementioned biomembrane phase-change droplet (PCD) and drug carrier.

### CROSS REFERENCE TO RELATED APPLICATIONS

This application is based upon and claims the benefit of priority from the prior Taiwan Patent Application No. 104113844, filed on Apr. 30, 2015, the entire contents of which are incorporated herein by reference.

### BACKGROUND OF THE INVENTION

1. Field of the Invention

The present disclosure relates to a phase-change droplet (PCD), and in particular it relates to phase-change droplet (PCD) encapsulated by a biomembrane, which is capable of carrying drugs.

2. Description of the Related Art

Erythrocytes (red blood cells; RBCs) are the most common type of blood cell in the blood. They have developed into delivery carriers having a long circulatory time in the nature, which carry oxygen to the parts of the body through the blood circulatory system. Flexible membranes, good physical and chemical stability, and special surface recognition molecules enable red blood cells to successfully transport oxygen in the circulatory system without being attacked by the immune system or affected by biomolecules, or alternatively, not being blocked or ruptured while transporting through narrow capillaries. Therefore, it is very suitable for red blood cells being used as carriers to transport oxygen in the circulatory system.

The main factor that prevents red blood cells from being recognized by the immune system, thereby prolonging the life in the circulatory system, is that the red blood cells have many different types of self-markers on the membrane. These self-marker proteins which are capable of conducting immune regulation prevent the red blood cells from the complement reaction or being recognized and attacked by macrophages. Therefore, red blood cells have a good circulatory stability in vivo.

In mammals, red blood cells are 6-8 μm in length and about 2 μm in thickness. Moreover, they differentiate into double concave discs without nucleus or organelles to obtain a maximum loading of hemoglobin and a minimum nutrient consumption. Typically, the life of human red blood cells is 100-120 days (about 30-40 days for mice). The total transport mileage is about 250 km in the blood circulatory system, which is superior to that of the drug carriers of current design (for example, PEG modified liposomes have a circulatory time of about dozens of hours).

Due to their inherent good characteristics such as biocompatibility, biodegradability, and not inducing immune reactions, the red blood cells have a great success in being applied to design drug carriers using the aforementioned good characteristics. For example, cargo laden red blood cells, synthetic carriers imitating red blood cells, red blood cell membrane derived liposomes, and red blood cell membrane camouflaged nanoparticles, etc.

However, both of the traditional drug carriers and the current RBC-derived drug carriers belong to a passive drug release mode, which conduct passive diffusion release by drug concentration gradient or carrier stability and result in a low drug release efficacy and difficulties in controlling the releasing position, dosage and release time. However, carriers which can be controlled by a trigger improve upon such disadvantages, of which the remote trigger control has received the most attention.

A drug delivering system using a remote trigger for release can control the drug releasing position, timing, lasting time, or dosage, etc. Along with a non-invasive trigger source such as light, a magnetic, an electric source, or an ultrasonic source, the behavior of the drug release can be controlled by conducting a remote trigger to the drug carriers.

Except for delivering drugs to target position, it is also expected to release drug at the target position at the proper time using drug carriers. Accordingly, the biocompatibility of the drug carrier itself and the method of releasing the drug is very important. Therefore, a drug carrier has both good biocompatibility and the ability for the drug release be triggered remotely is desired.

### BRIEF SUMMARY OF THE INVENTION

In one embodiment, the present disclosure provides a biomembrane phase-change droplet (PCD), including a hydrophobic liquid core; and a phospholipid-containing biomembrane encapsulating the hydrophobic liquid core, wherein the hydrophobic liquid core is vaporized by ultrasonic irradiation.

In another embodiment, the present disclosure also provides a drug carrier, including a biomembrane phase-change droplet; and a hydrophobic drug embedded on the biomembrane of the biomembrane phase-change droplet, wherein the hydrophobic drug is presented in an amount of 1-10 wt %, based on the weight of the drug carrier.

In still another embodiment, the present disclosure further provides the use of the biomembrane phase-change droplet as set forth above as an ultrasound contrast agent.

In still another embodiment, the present disclosure further provides the use of the biomembrane phase-change droplet as set forth above to manufacture a medicine for cancer therapy.

A detailed description is given in the following embodiments with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the subsequent detailed description and examples with references made to the accompanying drawings, wherein:

FIG. 1A is a cross section of a biomembrane phase-change droplet according to an embodiment of the present disclosure.

FIG. 1B illustrates a cross-sectional view of the biomembrane phase-change droplet before and after the high-intensity focused ultrasound (HIFU) radiation according to the embodiments of the present disclosure.

FIG. 2A is a cross section of a drug carrier according to another embodiment of the present disclosure.

FIG. 2B illustrates a cross-sectional view of the drug carrier before and after the high-intensity focused ultrasound (HIFU) radiation according to the embodiments of the present disclosure.

FIGS. 3A-3C illustrate the surface morphology of gold nanoparticles before, after the modification of silicon oxide, and after a further fluorocarbonization.

FIG. 3D illustrates the UV-visible spectrum of gold nanoparticles before, after the modification of silicon oxide, and after a further fluorocarbonization.

FIG. 4 illustrates the correlation of the gold nanoparticle content of the resulting biomembrane phase-change droplets and the different concentrations of gold nanoparticles added during the manufacturing process.

FIG. 5 illustrates a TEM image of fluorocarbon gold nanoparticles loaded into murine red blood cell membrane phase-change droplets (RBCMD).

FIGS. 6A-6D illustrate the surface morphology and particle size distribution of the murine red blood cell membrane phase-change droplets (RBCMD) loaded without, with 100 μg/mL, 200 μg/mL, and 400 μg/mL of camptothecin (CPT).

FIGS. 6E-6F respectively illustrate the surface morphology and particle size distribution of the murine red blood cell membrane phase-change droplets (RBCMD) loaded with fluorocarbon iron oxide nanoparticles.

FIG. 6G illustrates the surface morphology of the murine red blood cell membrane phase-change droplets (RBCMD) loaded with fluorocarbon gold nanoparticles.

FIG. 7 illustrates the analyzed result of the membrane protein retention of the murine red blood cell membrane phase-change droplets (RBCMD).

FIG. 8 illustrates the quantized result of uptake of droplets by macrophages, which is analyzed by flow cytometry.

FIG. 9 illustrates the analyzed result of the membrane protein retention of fresh/10 days-old murine red blood cell membrane (RBCM) and the murine red blood cell membrane phase-change droplets (RBCMD).

FIG. 10 illustrates the microscopic image of the murine red blood cell membrane phase-change droplets (RBCMD) before and after the acoustic droplet vaporization (ADV).

FIG. 11 illustrates the microscopic image of the fluorocarbon iron oxide nanoparticles-loaded murine red blood cell membrane phase-change droplets (RBCMD) before and after the acoustic droplet vaporization (ADV).

FIG. 12 illustrates the microscopic image of the fluorocarbon gold nanoparticles loaded murine red blood cell membrane phase-change droplets (RBCMD) before and after the acoustic droplet vaporization (ADV).

FIGS. 13A-13C respectively illustrate the analyzed result of drug releasing concentration, drug releasing efficacy, and inhibition of cell viability with/without ultrasonic irradiation.

FIGS. 14A and 14B respectively illustrate the B-mode image and quantified SNR value of the murine red blood cell membrane phase-change droplets (RBCMD) before and after the acoustic droplet vaporization (ADV).

FIGS. 15A and 15B respectively illustrate the microscopic image and cell viability of the pure cancer cells (BJAB) and in the presence of the murine red blood cell membrane phase-change droplets (RBCMD) before and after the ultrasonic irradiation.

FIG. 16 illustrates the B-mode image of the murine red blood cell membrane phase-change droplets (RBCMD) in mice after the ultrasound radiation.

FIGS. 17A and 17B respectively illustrate the surface morphology and particle size distribution of the human red blood cell membrane phase-change droplets (RBCMD) loaded without and with camptothecin (CPT).

FIGS. 18A and 18B illustrate the analyzed result of the membrane protein retention of the human red blood cell membrane phase-change droplets (RBCMD).

FIG. 19 illustrates the microscopic image of the human red blood cell membrane phase-change droplets (RBCMD) before and after the acoustic droplet vaporization (ADV).

FIGS. 20A-20C respectively illustrate the analyzed result of drug releasing concentration, drug releasing efficacy, and inhibition of cell viability with/without ultrasonic irradiation.

FIGS. 21A and 21B respectively illustrate the B-mode image and quantified SNR value of the human red blood cell membrane phase-change droplets (RBCMD) before and after the acoustic droplet vaporization (ADV).

FIGS. 22A and 22B respectively illustrate the microscopic image and cell viability of the pure cancer cells (BJAB) and in the presence of the human red blood cell membrane phase-change droplets (RBCMD) before and after the ultrasonic irradiation.

FIG. 23 illustrates the results of the dispersion test of gold nanoparticles before, after the modification of silicon oxide, and after a further fluorocarbonization.

FIG. 24 illustrates attraction of the iron oxide nanoparticles by a magnet.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The present disclosure utilizes a biomembrane having a phospholipid and self-identification molecules to encapsulate the liquid core which is capable of being vaporized by ultrasonic irradiation, thereby forming a biomembrane phase-change droplets with good biocompatibility an ability to reduce immune system attack. The bubbles generated from the vaporization of the liquid core can be used as an ultrasound contrast agent to enhance the ultrasound development. The present disclosure also utilizes the aforementioned biomembrane phase-change droplets loading with drugs to form drug carriers. Drug release of the drug carriers can be promoted by ultrasonic irradiation.

At the same time, the drug carriers which are capable of being released by remotely triggering can also serve as an ultrasound contrast agent, expecting developing medicines for treating diseases in clinical application.

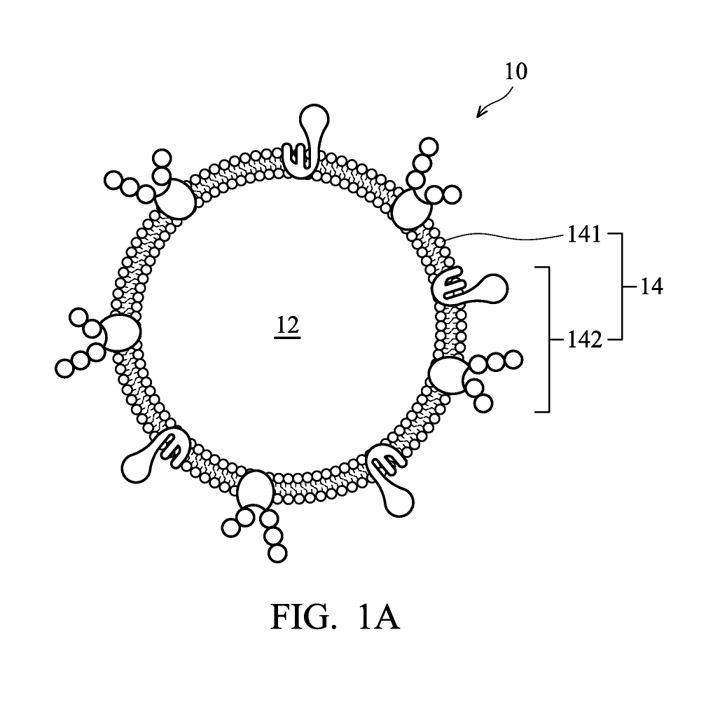

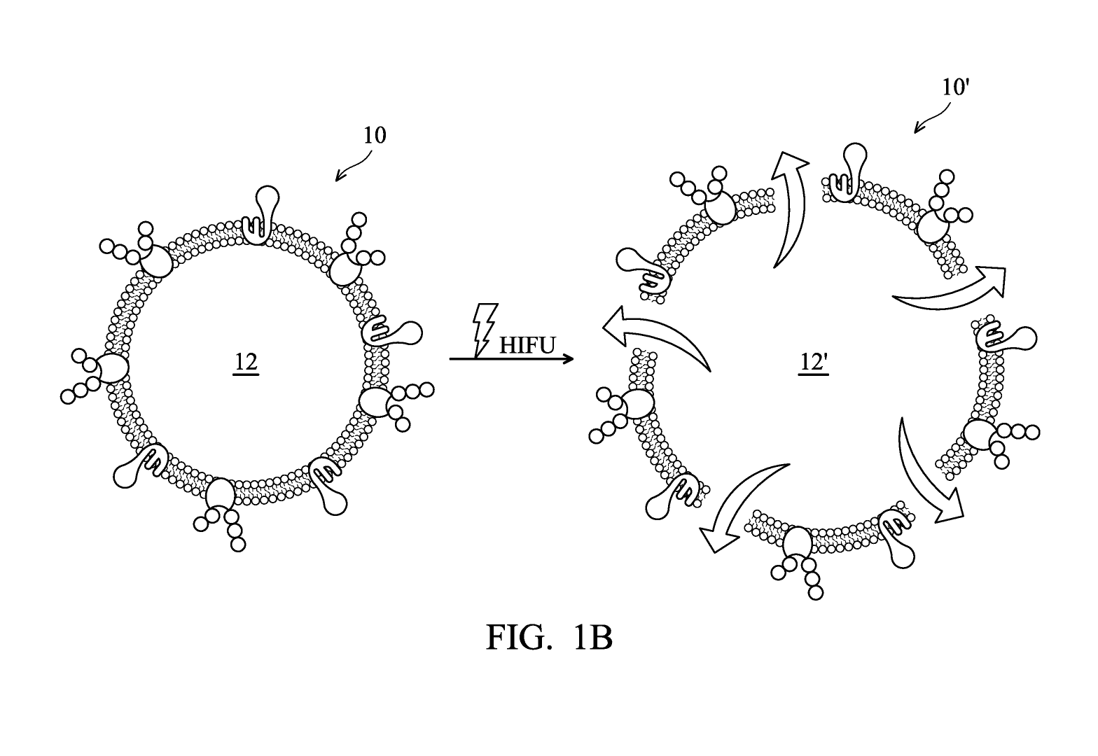

FIG. 1A is a cross section of a biomembrane phase-change droplet 10 according to an embodiment of the present disclosure. The biomembrane phase-change droplet 10 includes a hydrophobic liquid core 12 and a phospholipid 141-containing biomembrane 14 encapsulating the hydrophobic liquid core 12. The hydrophobic liquid core 12 has a low boiling point, for example, the boiling point of C₅F₁₂ or C₆F₁₄ is about 25° C.-60° C., which is vaporized by ultrasonic irradiation. The weight ratio of the hydrophobic liquid core 12 and the biomembrane 14 may be 1-20: 20-1, for example, 1-10: 10-1.

The hydrophobic liquid core 12 may include, for example, a fluorocarbon, other hydrophobic liquids, or a combination thereof. The fluorocarbon may include C₃F₈, C₄F₁₀, C₅F₁₂, C₆F₁₄, or a combination thereof. However, the hydrophobic liquid core 12 used in the present disclosure is not limited to the above. As long as capable of being vaporized by ultrasonic irradiation and remain a liquid state at room temperature, any hydrophobic liquids may serve as the hydrophobic liquid core 12. The ultrasound used to vaporize the hydrophobic liquid core 12 may include a high-intensity focused ultrasound (HIFU).

The biomembrane 14 of the biomembrane phase-change droplet 10 as shown in FIG. 1A may include red blood cell membrane, stem cell membrane, or other animal cell membrane having phospholipid bilayer structure. Cell membrane derived from mammals (for example, mice or human) themselves is preferred. The biomembrane 14 may include other membrane protein 142 such as glycoproteins, channel proteins, and different types of self-maker proteins, etc., other than phospholipid 141. The various types of self-marker proteins are capable of conducting immune regulation that prevent the biomembrane from the complement reaction or being recognized and attacked by macrophages in vivo, thereby helping the biomembrane phase-change droplet (PCD) to have a good circulatory stability in vivo.

FIG. 1B illustrates a cross-sectional view of the biomembrane phase-change droplet 10 before and after the high-intensity focused ultrasound (HIFU) irradiation according to the embodiments of the present disclosure. As shown in FIG. 1B, before the ultrasonic irradiation, the particle size distribution of the biomembrane phase-change droplet 10 may be between 0.1-5 μm, for example, 1-3 μm, or 2-3 μm. After the ultrasonic irradiation, such as a high-intensity focused ultrasound (HIFU) irradiation of 3.5 MHz, the hydrophobic liquid core 12 is vaporized and forms a biomembrane phase-change droplet 10′ having a larger particle size and volume, wherein the particle size is several times larger than before vaporization and the volume is dozens of times larger than before vaporization. The volume expansion phenomenon is accompanied by a physical blasting power and formation of bubbles with large volume. Therefore, the present disclosure also provides the use of the biomembrane phase-change droplets as an ultrasound contrast agent to enhance the ultrasound development.

In another embodiment, the biomembrane phase-change droplet 10 may further include a fluorocarbon nanoparticle, dispersing in the hydrophobic liquid core 12. The weight percentage of the fluorocarbon nanoparticle may be 0.1-5 wt %, such as 0.5 wt %, based on the weight of the hydrophobic liquid core. The fluorocarbon nanoparticles may include fluorocarbon iron oxide nanoparticles, fluorocarbon gold nanoparticles, fluorocarbon silicon oxide nanoparticles, or a combination thereof. It should be noted that, while the aforementioned fluorocarbon nanoparticles are fluorocarbon iron oxide nanoparticles, the biomembrane phase-change droplet 10 may be guided to specific position by the assistance of the magnetism of the iron oxide nanoparticles, and while the aforementioned fluorocarbon nanoparticles are fluorocarbon gold nanoparticles, the biomembrane phase-change droplet 10 may be triggered by IR irradiation.

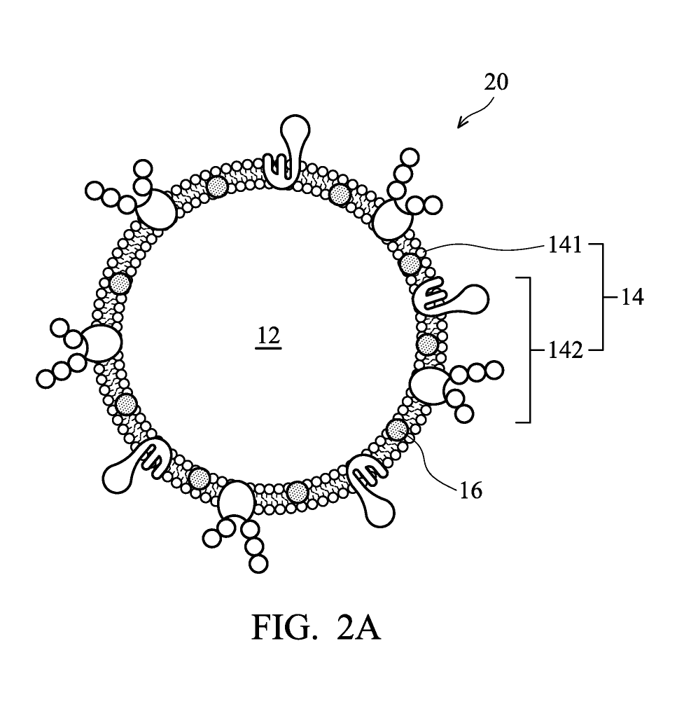

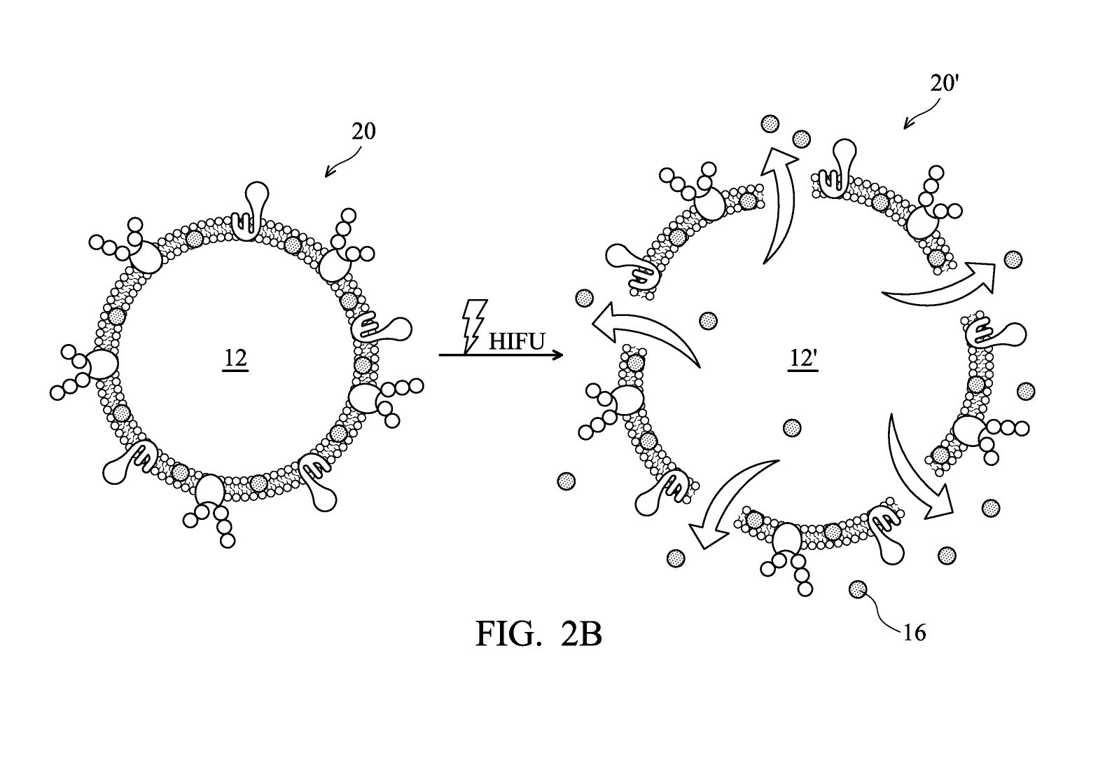

FIG. 2A is a cross section of a drug carrier 20 according to another embodiment of the present disclosure, the difference between the drug carrier and the aforementioned biomembrane phase-change droplet 10 is that the drug carrier 20 may further include a hydrophobic drug 16, embedded on the biomembrane 14 of the biomembrane phase-change droplets 10, wherein the hydrophobic drug 16 is presented in an amount of 1-10 wt %, for example, 6-4 wt %, based on the weight of the drug carrier 20.

The hydrophobic drug 16 may include, for example, camptothecin (CPT), paclitaxel, chlorin e6 (Ce6), or a combination thereof, but not limited the above disclosure. It should be noted that after the ultrasonic irradiation, the hydrophobic drug 16 would be released from the drug carrier 20 along with the vaporization of the hydrophobic liquid core 12 to achieve drug release which is remotely triggered by ultrasound.

FIG. 2B illustrates a cross-sectional view of the drug carrier 20 before and after the high-intensity focused ultrasound (HIFU) irradiation according to the embodiments of the present disclosure. As shown in FIG. 2B, before the ultrasonic irradiation, the particle size distribution of the drug carrier 20 may be between 0.1-5 μm, for example, 1-3 μm, or 2-3 μm. After the ultrasonic irradiation, such as a high-intensity focused ultrasound (HIFU) irradiation of 3.5 MHz, the hydrophobic liquid core 12 is vaporized and forms a drug carrier 20′ having a larger particle size and volume, wherein the particle size is several times larger than before vaporization and the volume is dozens of times larger than before vaporization. The volume expansion phenomenon is accompanied by a physical blasting power of bubbles with large volume. Therefore, the present disclosure also provides the use of the drug carrier as an ultrasound contrast agent to enhance the ultrasound development.

In addition, the present disclosure also provides the use of the drug carrier to manufacture a medicine for cancer therapy. According to different needs of diseases such as cancer, different drugs may be carried by the drug carriers which are remotely triggered by ultrasound to achieve the effect of treating different diseases.

In one embodiment, the biomembrane phase-change droplet 10 of the drug carrier 20 may further include a fluorocarbon nanoparticle, dispersing in the hydrophobic liquid core 12 of the biomembrane phase-change droplet 10. The weight percentage of the fluorocarbon nanoparticle may be 0.1-5 wt %, such as 0.5 wt %, based on the weight of the hydrophobic liquid core 12. The fluorocarbon nanoparticles may include fluorocarbon iron oxide nanoparticles, fluorocarbon gold nanoparticles, fluorocarbon silicon oxide nanoparticles, or a combination thereof. It should be noted that, while the aforementioned fluorocarbon nanoparticles are fluorocarbon iron oxide nanoparticles, the drug carrier 20 may be guided to specific position or conduct a magnetic thermal treatment by the assistance of the magnetism of the iron oxide nanoparticles, and while the aforementioned fluorocarbon nanoparticles are fluorocarbon gold nanoparticles, the drug carrier 20 may be triggered by IR irradiation or conduct a photothermal therapy.

The present disclosure mainly utilizes nature, self-available biomembrane as the encapsulating material to encapsulate the liquid core which can be vaporized by ultrasound and further carry drugs. The biomembrane phase-change droplets have a good biocompatibility and physical stability due to the retention of most of the proteins on the original biomembrane, which can prevent recognition and attack from the immune system. Also, after the ultrasonic irradiation, the bubbles generated by the liquid can serve as an ultrasound contrast agent. Moreover, the carried drugs can be released with physical blasting power accompanied by the vaporization of the liquid due to the ultrasonic irradiation. Additionally, the released drugs still remain the original drug efficacy.

In addition, the biomembrane phase-change droplet of the present disclosure may further include fluorocarbon nanoparticles such as fluorocarbon iron oxide nanoparticles and fluorocarbon gold nanoparticles. The drug carriers made of the aforementioned biomembrane phase-change droplets may use the magnetism of the fluorocarbon iron oxide nanoparticles as a means to guide the drug carrier to specific position or conduct a magnetic thermal treatment, or implanting IR irradiation to the drug carrier with fluorocarbon gold nanoparticles to trigger the drug carrier to release drug or conduct photothermal therapy.

To explore the related properties and application potential of the biomembrane phase-change droplet, the following description takes red blood cell membrane (RBCM) as an example to manufacture the red blood cell membrane phase-change droplets (RBCMD) and observe the appearance of the droplets, test the particle size distribution of the droplets, analyze the membrane protein retention, determine the drug loading efficacy, conducting macrophages swallow test, high-speed image observation of the ultrasound triggered droplet vaporization, examine the B-mode image, evaluate the damage to cancer cells of the ultrasound triggered drug release, and evaluate the physical damage to cancer cells of the ultrasound triggered droplet vaporization. Furthermore, the invention may test the effect of the ultrasound triggered droplet vaporization in vivo (mice) to evaluate the feasibility in vivo application.

In addition, the present disclosure not only forms phase-change droplets by the use of a murine red blood cell membrane, but also tests the basic properties of the phase-change droplets formed by the use of a human red blood cell membrane, expecting the ultrasound triggerable red blood cell membrane phase-change droplets can have more customized clinical potential.

### Materials and Methods

### Materials

| Material | Company | CAS No. |
|---|---|---|
| Ethylenediaminetetraacetic acid | J. T. Baker | 6381-92-6 |
| (EDTA) | | |
| Perfluoro-n-pentane (PFP; C₅F₁₂) | Strem Chemicals | 678-26-2 |
| Camptothecin (CPT) | Sigma-Aldrich | 7689-03-4 |
| 3,3′-Dioctadecyloxacarbocyanine | Sigma-Aldrich | 34215-57-1 |
| perchlorate (DiO) | | |
| Dimethyl sulfoxide (DMSO) | J. T. Baker | 67-68-5 |
| 40% acrylamide/Bis solution | MDBio | 79-06-1, 110-26-9 |
| Ammonium persulfate | MDBio | 7727-54-0 |
| Tetramethylethylenediamine | usb | 110-18-9 |
| (TEMED) | | |
| Methanol, HPLC grade | ECHO | 67-56-1 |
| Thiazolyl blue tetrazolium bromide | AMRESCO | 298-93-1 |
| (MTT) | | |
| 1,2-Distearoyl-sn-glycero-3- | Genzyme | 816-94-4 |
| phosphocholine (DSPC) | | |
| MPEG-2000-DSPE | Genzyme | 147867-65-0 |

### Extraction and Purification of the Murine Red Blood Cell Membrane

The method for the extraction and purification of the murine red blood cell membrane refers to the published article (Hu, C. M. J., et al., Proceedings of the National Academy of Sciences of the United States of America, 2011. 108(27): p. 10980-10985) with slight modifications. After anesthetizing mice, the heart blood was obtained by syringes with ethylenediaminetetraacetic acid (EDTA) solution. The obtained blood was kept in Vacutainer® (BD Biosciences) and mixed evenly. Anticoagulant containing whole blood was centrifuged, and the supernatant and white blood cell layer (Buffy coat) were removed. Sedimented red blood cells were resuspended/washed with PBS. Centrifugation/washing steps were repeated three times. Washed red blood cells were treated with hypotonic solution to release cytoplasmic components. The purified murine red blood cell membranes were obtained after three rounds of centrifugation/resuspension. A portion of the purified murine red blood cell membranes were freezed and dried, then weighed to estimate the concentration of the obtained red blood cell membranes.

### Extraction and Purification of the Human Red Blood Cell Membrane

The human blood samples used in the present disclosure conform to the rule of acquisition and use of National Tsinghua University. Blood collected from the vein of healthy volunteers was put into EDTA containing blood collection tube. Anticoagulant containing whole blood was centrifuged, and the supernatant and white blood cell layer (Buffy coat) were removed. Sedimented red blood cells were resuspended/washed with PBS. Centrifugation/washing steps were repeated three times. The washed red blood cells were treated with hypotonic solution to release cytoplasmic components. The purified murine red blood cell membranes were obtained after three rounds of centrifugation/resuspension. A portion of the purified murine red blood cell membranes were freezed and dried, then weighed to estimate the concentration of the obtained red blood cell membranes.

### Cell Cultivation

BJAB cells were cultured in RPMI medium with 10% Fetal bovine serum (FBS) and were sub-cultured every three days. Hela cells were cultured in DMEM medium with 10% Fetal bovine serum (FBS) and was sub-cultured every three days.

### Preparation Example 1

### Murine Red Blood Cell Membrane Phase-Change Droplets

1.94 ml of the purified murine red blood cell membrane solution, 0.06 ml of glycerol and 0.28 ml perfluoropentane (C₅F₁₂) were mixed on ice. Droplets with fluorescent dye (3,3′-dioctadecyloxacarbocyanine perchlorate; DiO) were dissolved in dimethyl sulfoxide (DMSO) and 0.05 ml of this solution was added to the mixed solution. This murine red blood cell membrane mixed solution was emulsified on ice by sonication using a probe type ultrasonic machine (Vibracell™, SONICS). Due to the ultrasonic energy, each ingredient was self-assembled to produce murine red blood cell membrane phase-change droplets. Emulsified droplets formed after sonication were washed three times with PBS to remove the unused components to obtain murine red blood cell membrane phase-change droplets.

### Preparation Example 2

### Drug Carrier—Murine Red Blood Cell Membrane Phase-Change Droplets Loaded with Drugs

The method is the same as Preparation Example 1, but the fluorescent dye (3,3′-dioctadecyloxacarbocyanine perchlorate; DiO) is replaced with an anticancer drug camptothecin (CPT) to obtain phase-change droplets loaded with camptothecin (CPT).

### Preparation Example 3

### Murine Red Blood Cell Membrane Phase-Change Droplets Loaded with Fluorocarbon Iron Oxide Nanoparticles

First, referring to the method of reference (J. Mater Chem B, 2014, 2, 1048) to producing silicon oxide-iron oxide nanoparticles. 0.005 g of the iron oxide nanoparticles were dispersed in 5 ml of toluene, then 0.04 ml of tetraethyl orthosilicate (TEOS) and 0.025 ml of triethylamine were added and reacted for 24 hours. Thereafter, the resulting silicon oxide-iron oxide nanoparticles were dispersed in 8 ml of methanol, and 0.09 ml of 1H, 1H, 2H, 2H-perfluoro heptadecane trimethyloxonium silane was added and uniformly mixed. Finally, 0.02 ml aqueous ammonia was added to obtain the fluorocarbon iron oxide nanoparticles.

Next, 1.94 ml of the purified murine red blood cell membrane solution, 0.06 ml of glycerol, 0.001 g of the fluorocarbon iron oxide nanoparticles, and 0.1 ml of the perfluoropentane (C₅F₁₂) were mixed on ice. This murine red blood cell membrane mixed solution was emulsified on ice by sonication using a probe type ultrasonic machine (Vibracell™, SONICS). Due to the ultrasonic energy, each ingredient was self-assembled to produce murine red blood cell membrane phase-change droplets. Emulsified droplets formed after the sonication were removed by washing three times with PBS to remove unused components to obtain biomembrane phase-change droplets loaded with fluorocarbon iron oxide nanoparticles.

### Preparation Example 4

### Murine Red Blood Cell Membrane Phase-Change Droplets Loaded with Fluorocarbon Gold Nanoparticles

First, referring to the method of reference (Nano Lett, Vol 8, No. 1, 2008) to producing silicon oxide-gold nanoparticles (silica-AuNR). Under continuous stirring, 100 μL of 0.1 M NaOH was slowly added to 10 ml of the gold nano-rod solution (concentration O.D.˜4) to adjust the pH value between 10 to 11. Then, 30 μL of 20% tetraethyl orthosilicate (TEOS) was added every 30 minutes three times. After being uniformly mixed, the TEOS used the protecting group CTAB (hexadecyl trimethyl ammonium bromide) of the surface of the nanoparticles as a substrate to conduct a hydrolysis-condensation reaction. After continuous stirring and reaction for two days, stable silicon oxide shell layer was formed on the gold nanoparticles. Next, high speed centrifugation was applied to remove excess TEOS and any remaining CTAB. Simultaneously, the reaction solvent was replaced with methanol. The re-dispersed silicon oxide-gold nanoparticles (silica-AuNR) were adjusted to a gold nano-rod solution of 3.5 ml, OD=4. Then, after 60 μL, of 1H, 1H, 2H, 2H-perfluoro heptadecane trimethyloxonium silane (1H, 1H, 2H, 2H-perfluorodecyltriethoxysilane) (fluoroalkylsilane; FAS) was added and uniformly mixed, 25 μL, of 30% aqueous ammonia was added to make the silane group and silicon dioxide conduct a hydrolysis condensation reaction to synthesis fluorocarbon gold nanoparticles.

Next, 855 μL of purified murine red blood cell membrane solution, 25 μL of glycerol, and 120 μL of the perfluoropentane (C₅F₁₂) containing fluorocarbon gold nanoparticles (O.D.=400) were mixed on ice. This murine red blood cell membrane mixed solution was emulsified on ice by sonication using a probe type ultrasonic machine (Vibracell™, SONICS). Due to the ultrasonic energy, each ingredient was self-assembled to produce murine red blood cell membrane phase-change droplets. Emulsified droplets formed after the sonication were removed by washing three times with PBS to obtain biomembrane phase-change droplets loaded with fluorocarbon gold nanoparticles.

### Analysis of the Appearance, Particle Size, and Characteristic of the Gold Nanoparticles

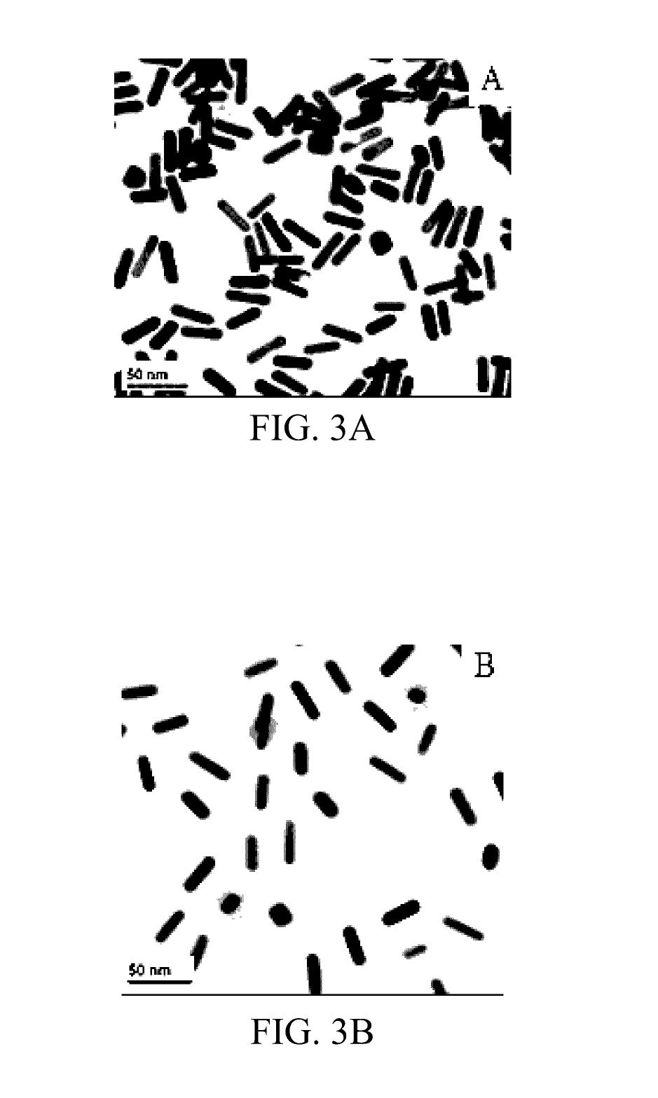

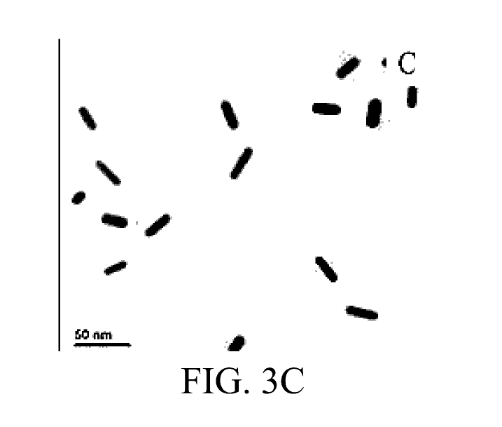

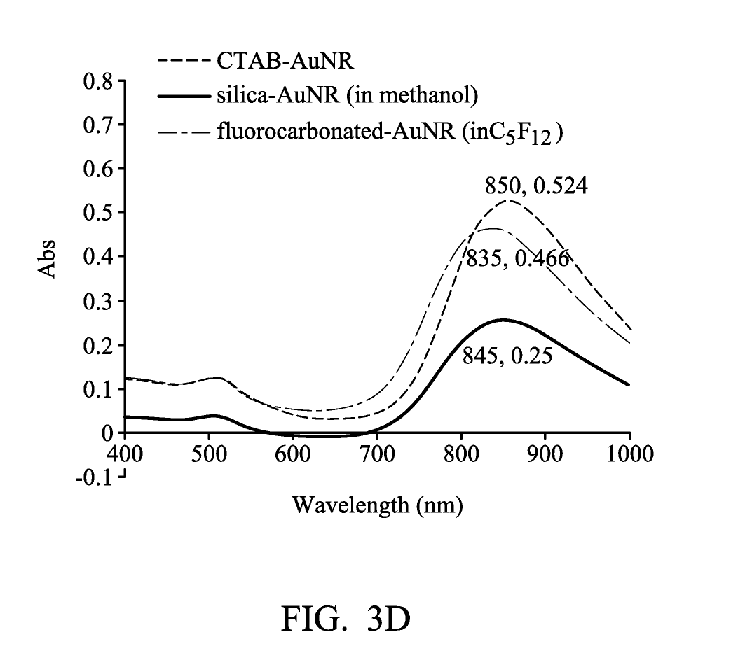

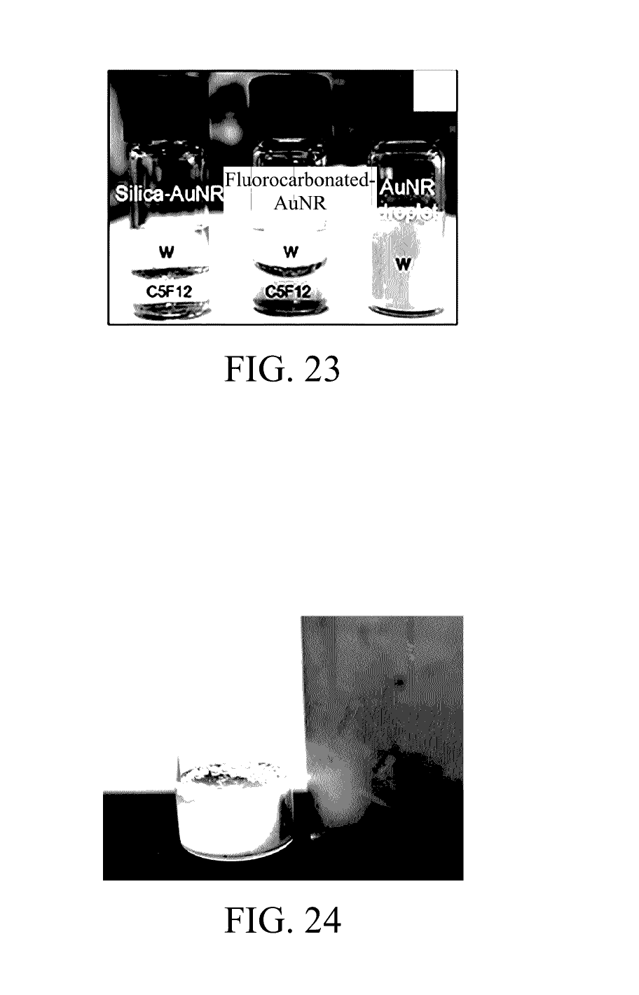

A transmission electron microscope (TEM) was used to observe the appearance and obtain photographs and images. FIGS. 3A-3C respectively show the surface appearance of the gold nanoparticles before and after the modification of silicon oxide, and the surface appearances of the further fluorocarbonated gold nanoparticles. As shown in FIG. 3A, the synthesized nano-gold particles have uniform structures. After modification of silicon oxide, it is clearly observed that the surface of the gold nanoparticles is covered with a layer of silicon oxide shell (FIG. 3B), and after fluorocarbonation, images of the transmission electron microscopy (TEM) does not show differences (FIG. 3C). However, in the dispersion test, it can be observed that gold nano-rods having a silicon oxide shell layer can disperse in the aqueous phase (w), whereas fluorocarbonated gold nano-rods can stably disperse in perfluoropentane (C₅F₁₂) (FIG. 23).

In addition, it can be found in the ultraviolet-visible spectroscopy (FIG. 3D) that after gold nanoparticles being modified by silicon oxide and fluorocarbonated, gold nanoparticles still retain the characteristics of surface electrical resonance, and the longitudinal absorption peak of the gold nano-rods has a blue displacement phenomenon.

### Determination of the Loading Content of the Gold Nanoparticles

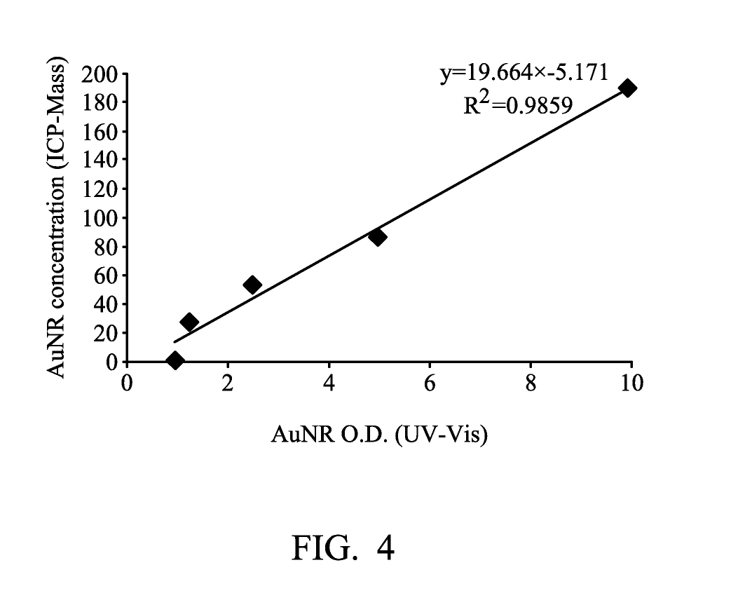

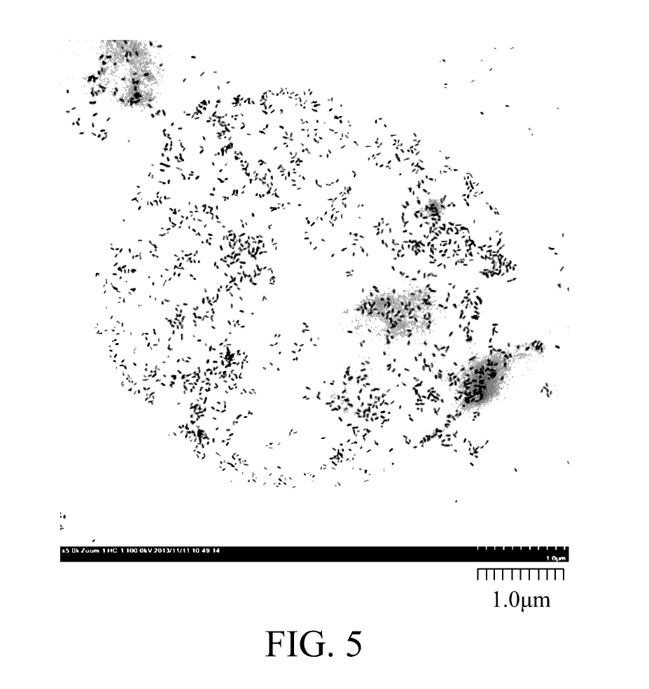

FIG. 4 illustrates the correlation of the gold nanoparticle content of the resulting biomembrane phase-change droplets and different concentration of gold nanoparticles added during the manufacturing process. According to FIG. 4, the concentrations of the added gold nanoparticles have a good linear correlation with the gold nanoparticles content in the resulting biomembrane phase-change droplets. The encapsulating ratio of the gold nanoparticles is 189.7 μg nano gold particle/2×10⁹ murine red blood cell membrane phase-change droplets (RBCMD) calculated by ICP-MASS and Multisizer. As shown in FIG. 5, it can be observed through TEM imaging that a large number of gold nanoparticles can be encapsulated within the murine red blood cell membrane phase-change droplets (RBCMD).

### Analysis of the Appearance and Particle Size of the Droplets

Ten-fold diluted murine red blood cell membrane phase-change droplets, phase-change droplets loaded with 100 μg/mL, 200 μg/mL, 400 μg/mL of camptothecin (CPT), phase-change droplets loaded with fluorocarbon iron oxide nanoparticles, and phase-change droplets loaded with fluorocarbon gold nanoparticles were each dropped on a slide, and a coverslip was placed gently at a slight angle. A fluorescent microscope (Observer D1, ZEISS) equipped with a camera system (AxioCam MRm) was used to observe the appearance and take pictures to obtain micrographs. The step involved conducting an analysis of the droplet size. 20 μL of each of the aforementioned droplet was diluted in 20 mL isotonic solution. Automated particle size analysis Coulter counter (Multisizer™ 3 COULTER COUNTER®, Beckman Coulter Inc., CA, USA) was used to analyze the particle size distribution and concentration of the droplets.

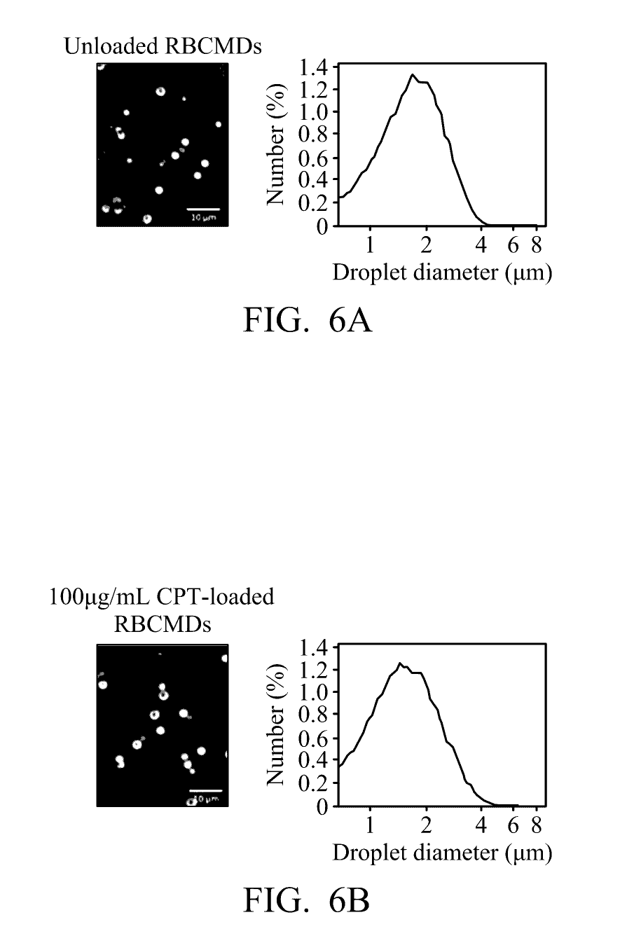

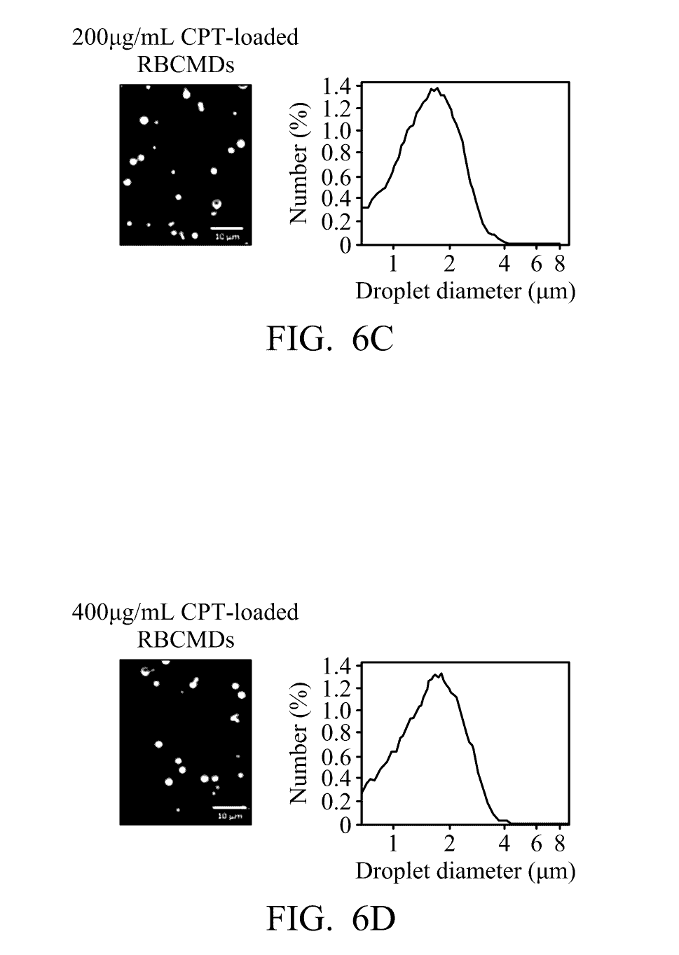

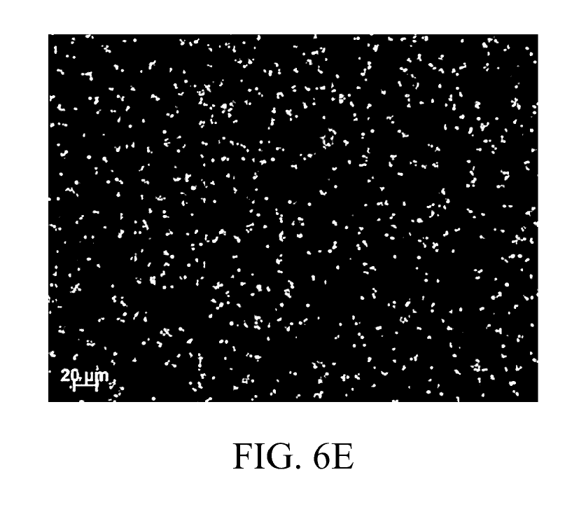

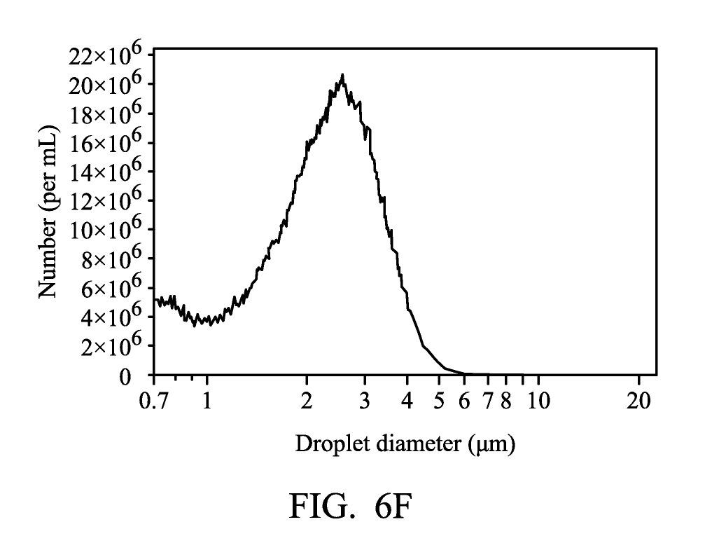

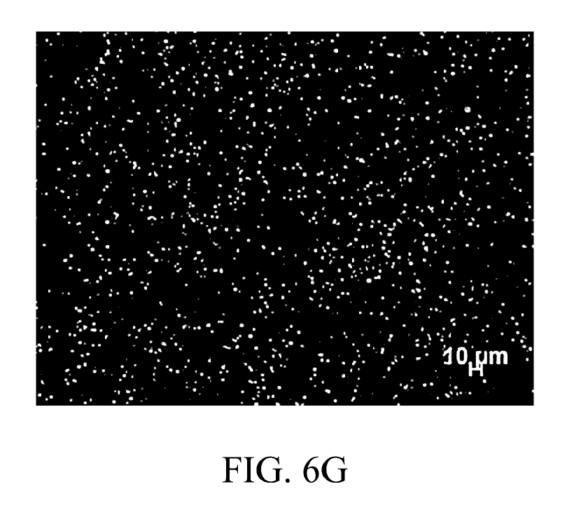

The results are shown in FIGS. 6A-6D, it can be found that each of the resulting droplets has spherical appearance, uniform shape, and good dispersion. The particle size of the droplets loaded with different concentrations of camptothecin has a bell-shaped distribution, and the main peak is mainly down to 1.7 μm. The concentration of droplet is approximately 1×10⁹ droplets/mL of whole blood.

FIGS. 6E-6F respectively illustrate the surface morphology and particle size distribution of the murine red blood cell membrane phase-change droplets (RBCMD) loaded with fluorocarbon iron oxide nanoparticles. It can be found that each of the resulting droplets has spherical appearance, uniform shape, and good dispersion. The particle size of the droplets has a bell-shaped distribution, and the main peak is mainly down to 2.3 μm.

Since containing iron oxide nanoparticles, the obtained droplets can be attracted by a magnet (FIG. 24).

FIG. 6G illustrates the surface morphology of the murine red blood cell membrane phase-change droplets (RBCMD) loaded with fluorocarbon gold nanoparticles. It can be found that each of the resulting droplets has a spherical appearance, uniform shape, and good dispersion. The particle size of the droplets has a bell-shaped distribution, and the main peak is mainly down to 2.1 μm.

### Analysis of the Retention of Red Blood Cell Membrane Protein

Extracted red blood cell membrane and resulting murine red blood cell membrane phase-change droplets (RBCMD) was added to loading buffer and then heated to 95° C. for 10 minutes to vaporize perfluoropentane (C₅F₁₂) and completely unfold the protein to linear. Then, the samples were analyzed by sodium dodecyl sulfate polyacrylamide gel electrophoresis (SDS-PAGE). Preparing two-layer polyacrylamide gel (10% and 18%) to conduct gel electrophoresis (Bio-RAD Mini-Protein Tetra System) to obtain a wide analysis range of protein molecular weight. 20 μL of sample containing loading buffer was loaded in gel to conduct gel electrophoresis under 100V for 150 minutes for protein separation. After electrophoretic separation, proteins were stained with Coomassie blue to analyze the protein position and protein composition and assess the retention of red blood cell membrane protein, as shown in FIG. 4. Table 1 shows the composition of SDS-PAGE:

TABLE 1

| | 18% separating | 10% | 5% |
|---|---|---|---|
| | gel | separating gel | stacking gel |
| H₂O | 2100 | 3600 | 3610 |
| 40% Acryl-bisacrylamide | 3375 | 1875 | 640 |
| 1.5M Tris buffer (pH8.8) | 1875 | 1875 | 0 |
| 1.5M Tris buffer (pH6.8) | 0 | 0 | 625 |
| 10% SDS | 75 | 75 | 50 |
| 10% Ammonium | 75 | 75 | 50 |
| persulfate | | | |
| TEMED | 5 | 5 | 5 |

Unit: μL

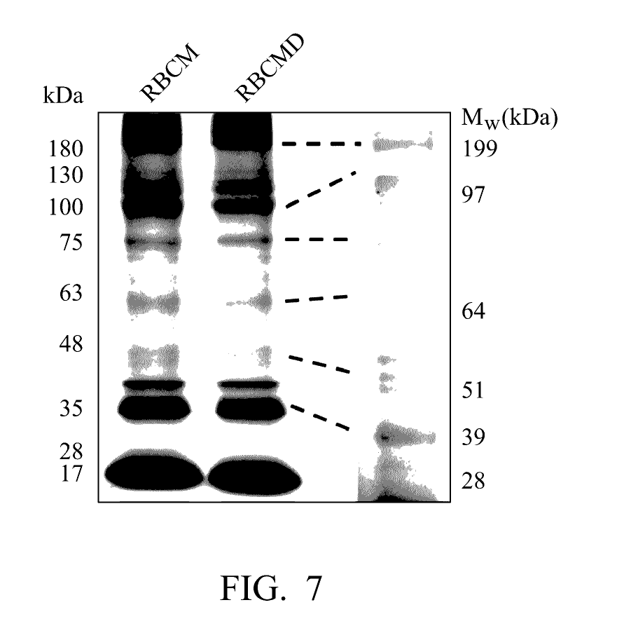

It is found in FIG. 7 that the protein composition of the murine red blood cell membrane phase-change droplets (RBCMD) and the initial red blood cell membrane (RBCM) is very close, revealing that most of the red blood cell membrane proteins are still successfully retained on the resulting murine red blood cell membrane phase-change droplets (RBCMD) after sonication. The rightmost column of FIG. 4 shows the composition of the murine red blood cell membrane proteins published in the reference (Hu, C M J, et al, Proceedings of the National Academy of Sciences of the United States of America, 2011. 108 (27): P 10980-10985), it can be found that the protein composition obtained in the present disclosure is similar to that shown in the reference.

### Analysis of the Drug Loading Efficacy

After the phase-change droplets loaded with different concentrations of camptothecin (CPT) was collapsed and dried, it was weighed and analyzed for the CPT content. Calculating the drug load efficiency (LE) and encapsulation efficiency (EE) corresponding to different initial added CPT concentrations.

It was found that the LE % of the phase-change droplets with initial CPT of 100 μg/mL is 0.87±0.16%, EE % of the phase-change droplets with initial CPT of 100 μg/mL is 102.55±12.57%. The LE % of the phase-change droplets with initial CPT of 200 μg/mL is 1.95±0.29%, EE % of the phase-change droplets with initial CPT of 200 μg/mL is 95.28±6.93%. The LE % of the phase-change droplets with initial CPT of 400 μg/mL is 3.37±0.72%, EE % of the phase-change droplets with initial CPT of 400 μg/mL is 80.87±6.19%. It reveals that with the rise of the initial drug concentration, LE % gradually increases (0.87% to 3.37%), while EE % gradually decreases (102.55% to 80.87%). Therefore, by according to these data, appropriate process can be selected to produce phase-change droplets with high efficacy of drug use (100 μg/mL, EE %=102.55%) or phase-change droplets with high loading (400 μg/mL, LE %=3.37%). The following experiments will use the phase-change droplets loaded with 100 μg/mL of camptothecin to conduct the test.

### Test of the Swallow of the Macrophages

First, murine primary peritoneal macrophages were isolated and cultured, the method for which is described in the reference (Zhang, X., R. Goncalves, and DM Mosser Curr Protoc Immunol, 2008. Chapter 14: P Unit 14 1.) with a slight adjustment. 10-week-old C57BL/6J black mice were intraperitoneally injected with 1 mL 3% thioglycolate broth to conduct a four-day induction of peritoneal macrophages. After anesthesia, blood from heart was purified to obtain fresh red blood cell membrane. Mice were sacrificed and the abdomen was opened, then 5 mL of PBS was intraperitoneal injected to suspend the macrophages in the abdomen. Next, the PBS solution mixed with cells was sucked out. After repeating three times, approximately 1×10⁷ cells can be obtained. 1×10⁶ cells/well were attached to the 6-well cell culture plate. Most of the cells being attachable to the plate are peritoneal macrophages

After overnight attachment, fluorescent targeted phase-change droplets respectively formed from fresh red blood cell membrane, old (placed at 4° C. for ten days) red blood cell membrane, synthesized lipid with PEG-modified, and synthesized lipid without PEG-modified made of fluorescent calibration phase variable droplet was co-cultured with peritoneal macrophages for 10 minutes and 60 minutes, respectively, then, washed with PBS three times. Cells were suspended by trypsin and fixed by 4% paraformaldehyde for 20 minutes. Thereafter, fluorescent state within the macrophages was analyzed by a flow cytometer, thereby analyze whether macrophages have different uptake level and efficiency towards different kinds droplets.

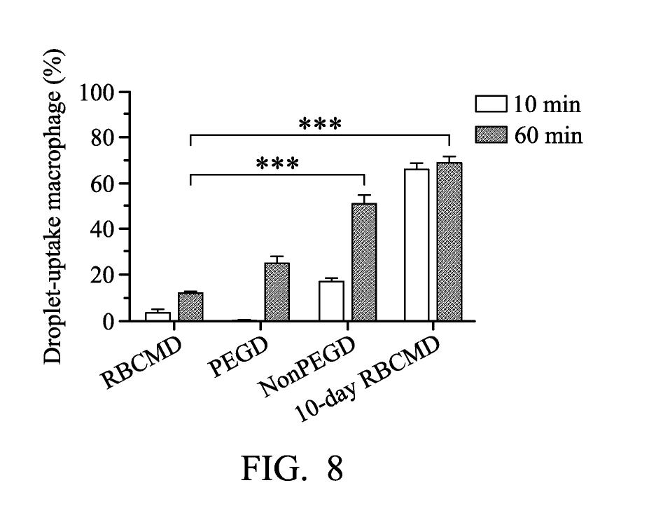

The results are shown in FIG. 8. It can be found that fresh murine red blood cell membrane phase-change droplets (RBCMD) and the PEG-modified lipid droplets (PEGD) were less uptaken. At 10 minutes, the uptake ratio is 3.1±1.4% and 0.3±0.2%, respectively. At 60 minutes, the uptake ratio is 11.5±1.1% and 24.2±3.2%, respectively. Non-PEG-modified lipid droplets (NonPEGD) show a higher uptake. At 10 minutes, the uptake ratio is 16.7±1.7%. At 60 minutes, the uptake ratio is 50.2±3.8%. Comparing to old (placed at 4° C. for ten days) murine red blood cell membrane (10-day RBCMD), old murine red blood cell membrane (10-day RBCMD) shows a highest uptake of macrophages. At 10 minutes, the uptake ratio is 65.5±2.6%. At 60 minutes, the uptake ratio is 68.2±2.6%.

It can be found that the freshly prepared murine red blood cell membrane phase-change droplets (RBCMD) have an effect of reducing swallow by macrophages compared to that of the PEG-modified lipid droplets. Both of them have quite significant differences between that of non-PEG-modified lipid droplets (NonPEGD) and old murine red blood cell membrane phase-change droplets.

### Analysis of the Retention of Red Blood Cell Membrane Protein

In order to verify whether the ability of significantly decreasing swallowed by macrophages is caused by the cell membrane protein loss or damage, membrane protein compositions of the fresh red blood cell membrane, the phase-change droplets formed thereof, the old red blood cell membrane, and the phase-change droplets formed thereof were analyzed by SDS-PAGE assay. Referring to FIG. 9, the result of membrane protein retention analysis reveals that the fresh red blood cell membrane (RBCM) and the phase-change droplets (RBCMD) formed thereof indeed retain more membrane protein compositions. Their protein broad bands are more significant. On the other hand, old red blood cell membrane (10-day RBCM) loses the short protein compositions of segments 130-180 kDa and 48-63 kDa. The phase-change droplets (10-day RBCMD) formed thereof retains fewer protein compositions, even the protein broad bands of segments 75 kDa and 100-130 kDa are disappeared. Comparing the results of protein retention analysis and the previous macrophages uptake experiments, it can be found that the two results have reasonable correlation. Therefore, it can be preliminarily deduced that the degree of membrane protein retention and the effect of reducing swallowed by macrophages are positively correlated.

High-Speed Image Observation of the Ultrasound Triggered Droplet Vaporization: Drug Carrier—Murine Red Blood Cell Membrane Phase-Change Droplets Loaded with Drugs

In order to verify the resulting red blood cell membrane phase-change droplets (RBCMD) were indeed loaded with perfluoropentane and could be vaporized by an ultrasonic trigger, the murine red blood cell membrane phase-change droplets were injected into micro-tube which is permeable to light and ultrasound, then a single ultrasonic irradiation was irradiated to the focus of the optical microscope to take images of the droplets before and after the irradiation of ultrasound by the high-speed camera. The high-speed images of the ultrasound triggered droplet vaporization were vaporized and observed by the photoacoustic confocal system. The system refers to the architecture used by P. A. Dayton, 1999 (Dayton, P A, et al, Ieee Transactions on Ultrasonics Ferroelectrics and Frequency Control, 1999. 46 (1): P 220-232) and is modified.

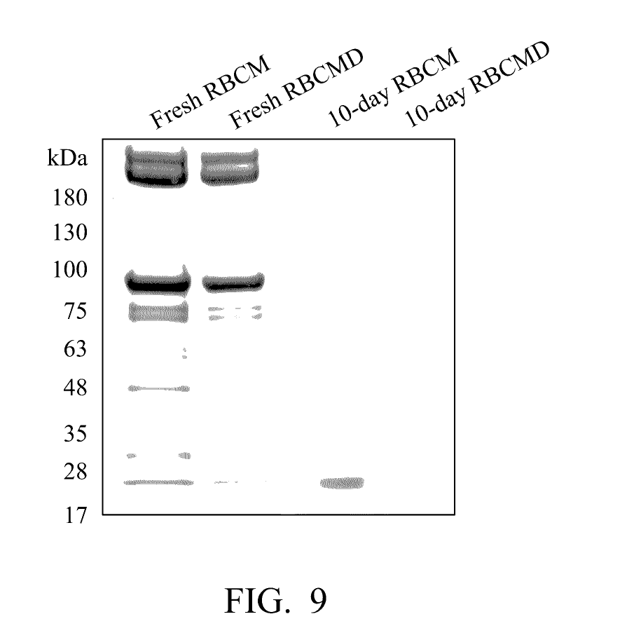

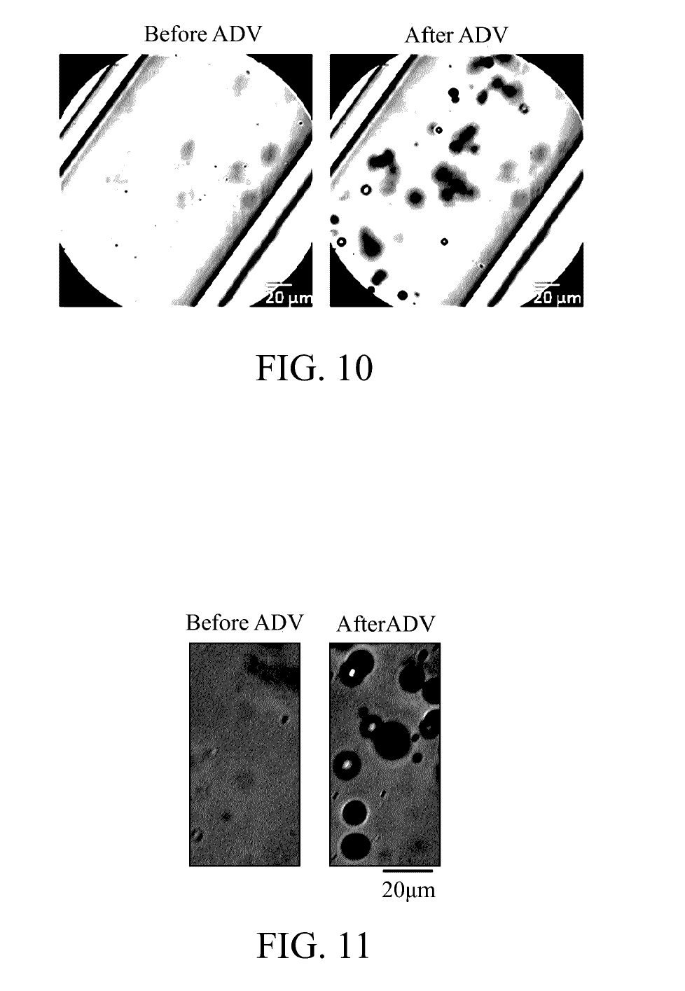

As shown in FIG. 10, before a single ultrasonic irradiation (before ADV), the murine red blood cell membrane phase-change droplets (RBCMD) appears to be spherical with small particle size. After ultrasonic irradiation (after ADV), lots of bubbles with particle sizes that were 5 times larger were produced, which is conformed with the theoretical size of vaporization from droplets to bubbles. It reveals that the resulting murine red blood cell membrane phase-change droplets (RBCMD) can be vaporized by single ultrasound trigger to form larger gas microbubbles having property of ultrasound triggered phase-change droplets. Also, it is confirmed that the resulting murine red blood cell membrane phase-change droplets (RBCMD) are successfully loaded with perfluoropentane.

High-Speed Image Observation of the Ultrasound Triggered Droplet Vaporization: Drug Carrier—Murine Red Blood Cell Membrane Phase-Change Droplets Loaded with Fluorocarbon Iron Oxide Nanoparticles

In order to verify the resulting biomembrane phase-change droplets loaded with fluorocarbon iron oxide nanoparticles could be vaporized by ultrasound trigger, the droplets were injected into micro-tube which is permeable to light and ultrasound, then a single ultrasonic irradiation was irradiated to the focus of the optical microscope to take images of the droplets before and after the irradiation of ultrasound by the high-speed camera. The high-speed images of the ultrasound triggered droplet vaporization were vaporized and observed by the photoacoustic confocal system. The system refers to the architecture used by P. A. Dayton, 1999 (Dayton, P A, et al, Ieee Transactions on Ultrasonics Ferroelectrics and Frequency Control, 1999. 46 (1): P 220-232) and is modified.

As shown in FIG. 11, before a single ultrasonic irradiation (before ADV), the murine red blood cell membrane phase-change droplets (RBCMD) appears to be spherical with small particle size. After ultrasonic irradiation (after ADV), lots of bubbles with particle sizes several times larger were produced, which conforms with the theoretical size of vaporization from droplets to bubbles. It reveals that the resulting droplets can be vaporized by single ultrasound trigger to form larger gas microbubbles having property of ultrasound triggered phase-change droplets.

High-Speed Image Observation of the Ultrasound Triggered Droplet Vaporization: Drug Carrier—Murine Red Blood Cell Membrane Phase-Change Droplets Loaded with Fluorocarbon Gold Nanoparticles

In order to verify that the resulting biomembrane phase-change droplets loaded with fluorocarbon gold nanoparticles could be vaporized by an ultrasonic trigger, the droplets were injected into a micro-tube which is permeable to light and ultrasound, then a single ultrasonic irradiation was irradiated to the focus of the optical microscope to take images of the droplets before and after the irradiation of ultrasound by the high-speed camera. The high-speed images of the ultrasound triggered droplet vaporization were vaporized and observed by the photoacoustic confocal system. The system refers to the architecture used by P. A. Dayton, 1999 (Dayton, P A, et al, Ieee Transactions on Ultrasonics Ferroelectrics and Frequency Control, 1999. 46 (1): P 220-232) and is modified.

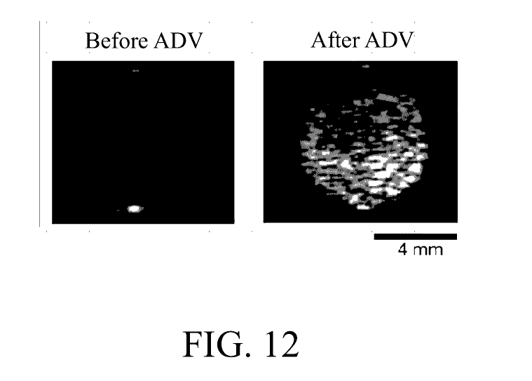

As shown in FIG. 12, before a single ultrasonic irradiation (before ADV), the murine red blood cell membrane phase-change droplets (RBCMD) appears to be spherical with small particle size. After ultrasonic irradiation (after ADV), lots of bubbles with particle sizes several times larger were produced, which conforms with the theoretical size of vaporization from droplets to bubbles. It reveals that the resulting droplets can be vaporized by a single ultrasonic trigger to form larger gas microbubbles having the properties of ultrasound triggered phase-change droplets.

### Evaluation of Damage to Cancer Cells of the Ultrasound Triggered Drug Release

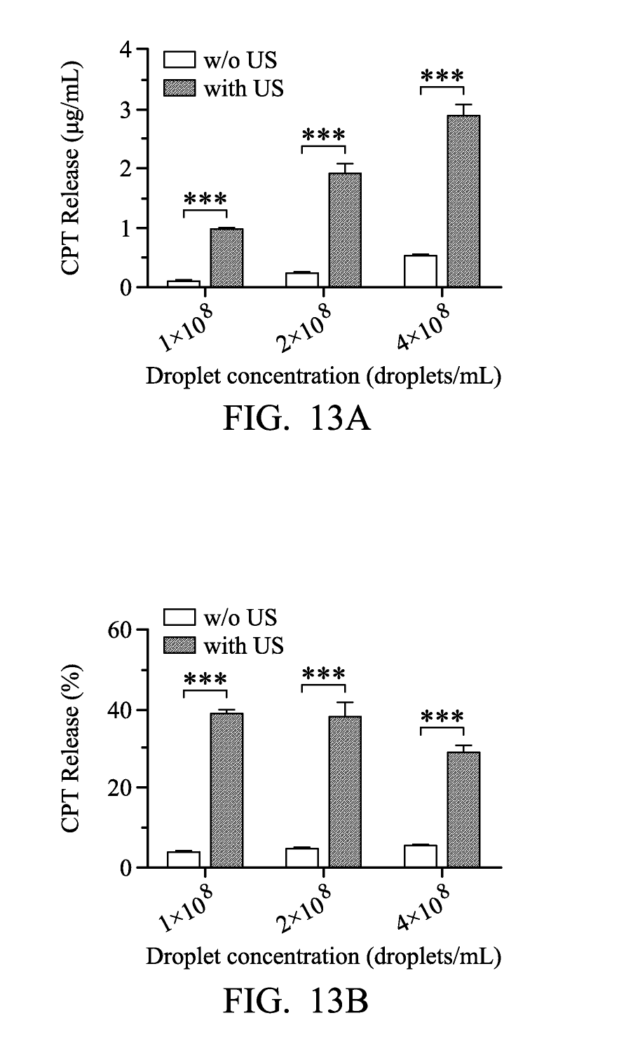

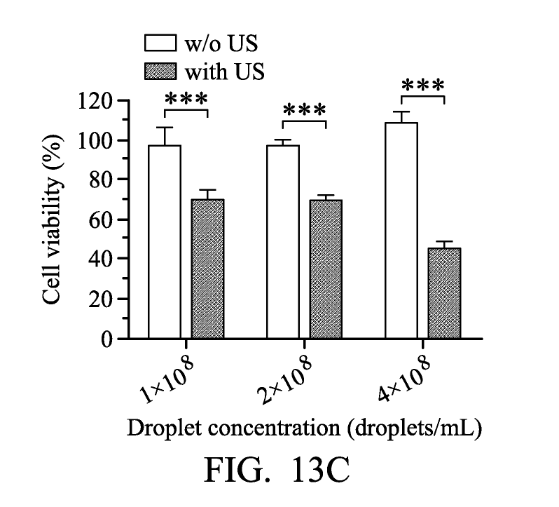

In order to verify whether the ultrasound triggered murine red blood cell membrane phase-change droplets (RBCMD) could be used as drug carriers whose release is ultrasonic remote controlled. Different numbers of red blood cell membrane phase-change droplets loaded with camptothecin were irradiated with HIFU three minutes, then centrifuged at 1500×g for 30 seconds to separate intact droplets. Supernatant containing drug was mixed with 50% (v/v) DMSO and 50% (v/v) PBS. The drug release efficacy was evaluated by the released concentration of camptothecin using fluorescent disc instrument. The results are shown in FIGS. 13A-13B.

FIG. 13A illustrates the significant differences of drug release concentration of different dosage red blood cell membrane phase-change droplets before and after HIFU irradiation. Under the concentration of 1×10⁸, 2×10⁸, and 4×10⁸ droplets/mL whole blood, the drug release concentration of non-irradiated groups are 0.09±0.01 μg/mL, 0.22±0.03 μg/mL and 0.52±0.03 μg/mL, respectively, whereas the drug release concentration of irradiated groups are 0.97±0.03 μg/mL, 1.90±0.15 μg/mL and 2.89±0.17 μg/mL, respectively. The ultrasound-triggered drug release concentration and the dosage of droplets have a positive correlation.

FIG. 13B illustrates the drug release efficacy of different droplets dosage after HIFU irradiation. The drug release efficacy of non-irradiated groups are all below 5%, whereas the drug release efficacy of irradiated groups are about 39% under the concentration of 1×10⁸ and 2×10⁸ droplets/mL whole blood. The drug release efficacy of higher concentration (4×10⁸ droplets/mL whole blood) is slightly decreased to 29%. The reason for this may be that, since droplets with a higher concentration cause more ultrasonic reflection and scattering, the drug release efficacy by a shadowing effect formed in the internal droplets is thereby reduced.

In order to verify whether the drug release triggered by ultrasound still has an anticancer effect, ultrasound triggered drug release sample was diluted 20-fold and co-cultured with cancer cells (Hela cells) for 24 hours, then a cell viability assay was conducted.

5×10⁴ cells/well were attached to 24-well plates and cultured for 24 hours. Ultrasound triggered released camptothecin solution was diluted 20-fold and added to culture medium of Hela cells. After being co-cultured with cancer cells for 24 hours, the culture medium was replaced with a fresh culture medium and MTT reagent was added. After a 4-hour culture, the culture medium and reagent were removed and cells were washed with PBS. 200 μL of DMSO was added to dissolve MTT metabolites. DMSO solution dissolved with MTT metabolites was transferred to 96-well transparent plate to analyze the absorbance value at 570 nm. Cell viability was calculated by the following formula: Cell viability=absorbance value of experimental group/absorbance value of control group (group only added PBS)×100%. Results are shown in FIG. 13C.

FIG. 13C shows that non-irradiated group does not have a significant effect on cancer cell viability (viability remains greater than 96%), whereas in the irradiated group, the cancer cell viability decreases to 69% under concentration of 1×10⁸ and 2×10⁸ droplets/mL whole blood, and the cancer cell viability further decreases to 44% under concentration of 4×10⁸ droplets/mL whole blood. It reveals that the camptothecin released by ultrasound trigger still has significant anticancer effect.

The above results confirm that murine red blood cell membrane phase-change droplets (RBCMD) can be triggered by ultrasound to promote drug release, and can be used as drug carriers which can be remotely triggered, and the drug release triggered by ultrasound still has an anticancer effect.

### In Vitro B-Mode Ultrasound Development

In order to further test the potential of the murine red blood cell membrane phase-change droplets as being an ultrasound contrast agent, B-mode ultrasound echo images differences between red blood cell membrane phase-change droplets with different dosages before and after the vaporization triggered by ultrasound are analyzed next. The system architecture for detecting the ultrasound reflective signals before and after the vaporization of phase-change droplets refers to the reference previously published by teacher Yeh, C. K. (Kang, S T; Yeh, C K Intracellular Acoustic Droplet Vaporization in a Single Peritoneal Macrophage for Drug Delivery Applications Langmuir 2011 27, 13183-13188).

First, phosphate buffer solution without phase-change droplets was loaded into agar gel phantoms, used as background signals of the system. After removing phosphate buffer solution, red blood cell membrane phase-change droplets with different concentrations (4×10⁶-64×10⁶ droplets/mL whole blood) were loaded into agar gel phantoms, and an ultrasound reflective signal before vaporization was captured first. After the droplets irradiated by HIFU for 5 seconds, the ultrasound reflection signals are captured again to detect the image signals after vaporization. After removing the background signals of the reflective signals of different droplets concentration and the reflective signals before and after vaporization, SNR values of each group were analyzed to evaluate the efficacy of red blood cell phase-change droplets used as an ultrasound contrast agent.

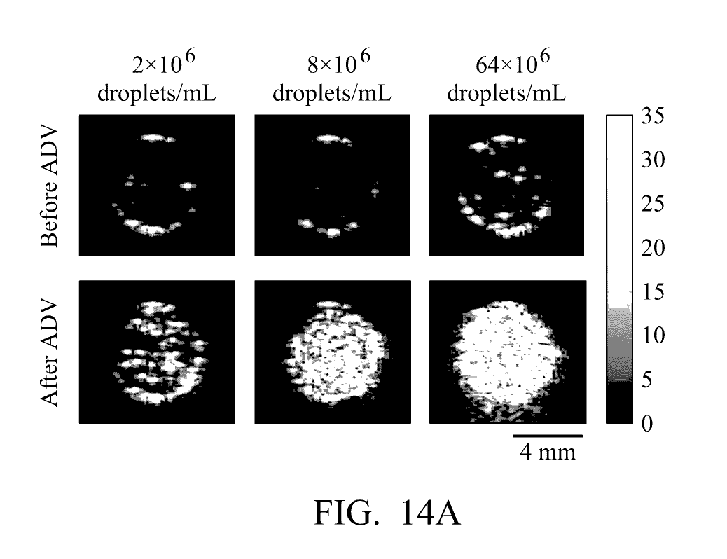

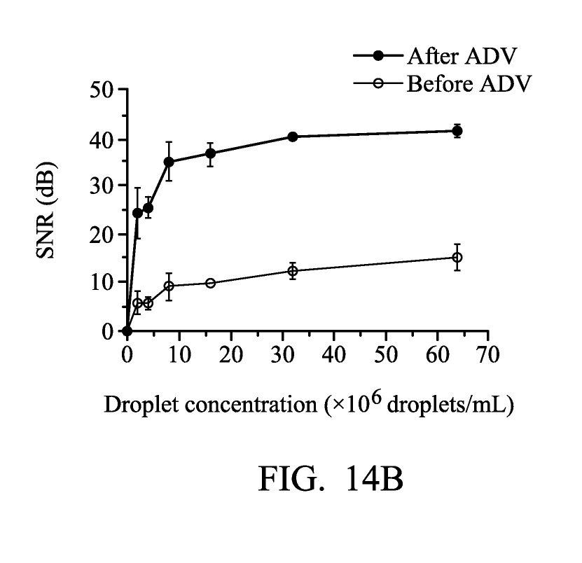

FIG. 14A illustrates the B-mode ultrasound echo images of the murine red blood cell membrane phase-change droplets in different concentrations before and after the acoustic droplet vaporization (ADV). It can be observed in the images that there are significant differences in B-mode image contrast before and after the ultrasound triggered vaporization. After vaporization, bubbles were produced and thereby the ultrasound reflective signals were enhanced and resulted in bright white images.

FIG. 14B illustrates diagram made of the quantified result of signal to noise ratio (SNR) of the ultrasound reflective signals. As the concentration of red blood cell phase-change droplets increases, the produced signal to noise ratio (SNR) of B-mode also gradually increases, the highest value can reach 41.5±1.3 dB. However, a saturation value was approached under a concentration of 32×10⁶ droplets/mL whole blood, continuous increase of dose cannot significantly enhance the reflected signals. Therefore, a concentration of 32×10⁶ droplets/mL whole blood or below is preferred. The B-mode images before and after the vaporization triggered by ultrasound reveal that murine red blood cell membrane phase-change droplets (RBCMD) indeed have the ability and potential to be used as an ultrasound contrast agent.

### Evaluation of Physical Damage to Cancer Cells of the Ultrasound Triggered Droplet Vaporization

The present disclosure then observes whether the blasting power caused by vaporization generated by ultrasound trigger to the murine red blood cell membrane phase-change droplets can destroy the adjacent targets in order to achieve the application of physically destroying adjacent cancer cells.

A plastic pipe having a window formed of polyvinylchloride (PVC) film was used as a sample compartment. Murine red blood cell membrane phase-change droplets (1×10⁹ droplets/mL whole blood) and BJAB cells (3×10⁴ cells/mL) were mixed and injected into the sample compartment. HIFU was irradiated through the window (references are the same as disclosed in the above vaporization experiments) three minutes to vaporize the murine red blood cell membrane phase-change droplets and ablate the peripheral cancer cells. In order to quantify and verify the damage towards adjacent cancer cells by the physical power generated by large level of droplet vaporization of, the lymphoma cells (BJAB cells) were mixed with murine red blood cell membrane phase-change droplets and added to gel ultrasound phantoms, then irradiated by HIFU to trigger vaporization. Vaporized cell mixed solution was collected and centrifuged to remove fragments.

Ultrasound-irradiated cell sample was collected and centrifuged at 300×g for 5 minutes. After removing the supernatant, the intact cells were resuspended in a fresh culture medium and transferred to a 24-well plate for microscopic image capture. After MTT reagent (1 mg/mL) was added and cultured with cells for 4 hours, culture medium and reagent were removed and cells were washed with PBS (centrifugation at 500×g for 5 minutes). 200 μL of DMSO was added to dissolve MTT metabolites. DMSO solution dissolved with MTT metabolites was transferred to 96-well transparent plate to analyze the absorbance value at 570 nm. Cell viability was calculated by the following formula: Cell viability=absorbance value of experimental group/absorbance value of control group (group only added PBS)×100%.

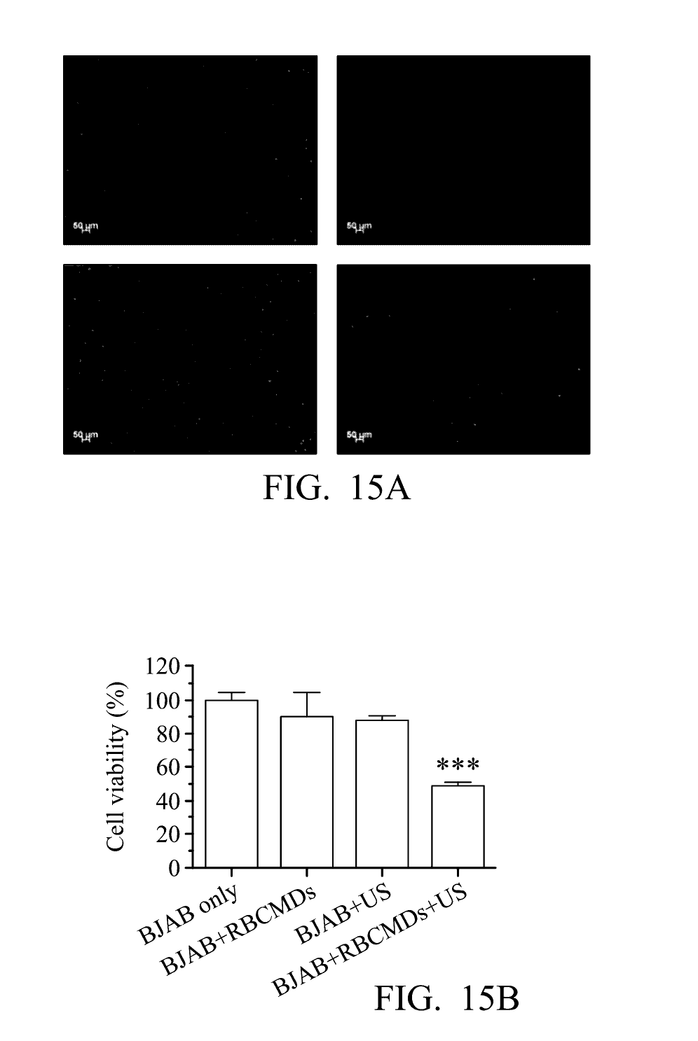

As shown in FIG. 15A, it is observed via microscope that the number of cancer cells (BJAB+RBCMDs+US) significantly decreased in the group with added murine red blood cell membrane phase-change droplets (RBCMD). Conversely, the control groups of pure cancer cells (BJAB), cancer cells with murine red blood cell membrane phase-change droplets (BJAB+RBCMD), and cancer cells with ultrasonic irradiation (BJAB+US) do not have significant differences in cell number. After conducting a cell viability assay, as shown in FIG. 15B, it can be found that the viability of the control group (non-irradiated) are still more than 90%, whereas the cell viability of groups with murine red blood cell membrane phase-change droplets and irradiated by ultrasound decreases to 49%. It reveals that murine red blood cell membrane phase-change droplets accompanied by the blasting power generated by ultrasound triggered vaporization can cause physical destruction of the adjacent target cells such as cancer cells and result in reduction of viability, thereby demonstrating the potential for application in cancer treatment and destruction of partial tumor blood vessel wall.

### Observation of the Ultrasound Triggered Droplet Vaporization in Mice

After investigating many properties and the application potential of murine red blood cell membrane phase-change droplets by in vitro experiments, the present disclosure further evaluates the performance of murine red blood cell membrane phase-change droplets in vivo. First, a test was conducted to determine whether droplets vaporization could be triggered in mice and enhance signal intensity of the ultrasound B-mode image as a developer.

### B-Mode Ultrasound Development in Mice

The droplets vaporization in mice and the ultrasound B-mode development were scanned by commercial ultrasound device (Aplio 500, Toshiba, Japan). Anesthetized mice with fur clipped were placed in HIFU/ultrasonic detection probe confocal system. A small amount of ultrasound contrast agent was orbital injected into blood circulation to confirm the confocal region and B-mode image. Stand for 20 minutes until the contrast-enhanced image disappeared. 50 μL of solution containing 5×10⁷ murine red blood cell membrane phase-change droplets was orbital injected into murine blood circulation. After irradiation for 2 minutes, B-mode image change in the confocal region was recorded.

FIG. 16 reveals that the B-mode signal was significantly enhanced after the HIFU irradiation (after ADV) compared to that before HIFU irradiation (before ADV). Experimental results reveal that murine red blood cell membrane phase-change droplets indeed circulates in vivo and produce droplets vaporization triggered by in vitro HIFU irradiation. Bubbles generated by droplets vaporization can enhance the B-mode image in time to be used in ultrasound development.

Hereinafter, the present disclosure further tests the phase-change droplets formed by human red blood cell membrane, including test the basic properties and the above experiments. The experimental methods are the same as that for the murine red blood cell membrane phase-change droplets. for the purpose of simplicity, the following description will not repeat the method, and merely shows the results as following:

### Preparation Example 5

### Human Red Blood Cell Membrane Phase-Change Droplets

The method for preparing human red blood cell membrane phase-change droplets is the same as described in Preparation Example 1, except that the murine red blood cell membrane is replaced with purified human red blood cell membrane to obtain the human red blood cell membrane phase-change droplets.

### Preparation Example 6

### Drug Carriers—Human Red Blood Cell Membrane Phase-Change Droplets Loaded with Drugs

The method for preparing drug carriers is the same as that of Preparation Example 5, except that the fluorescent dye (3,3′-dioctadecyloxacarbocyanine perchlorate; DiO) is replaced with an anticancer drug, camptothecin (CPT), to obtain phase-change droplets loaded with drugs.

### Analysis of the Appearance and Particle Size of the Droplets

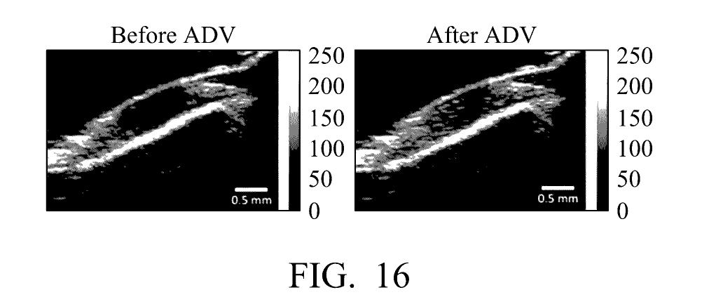

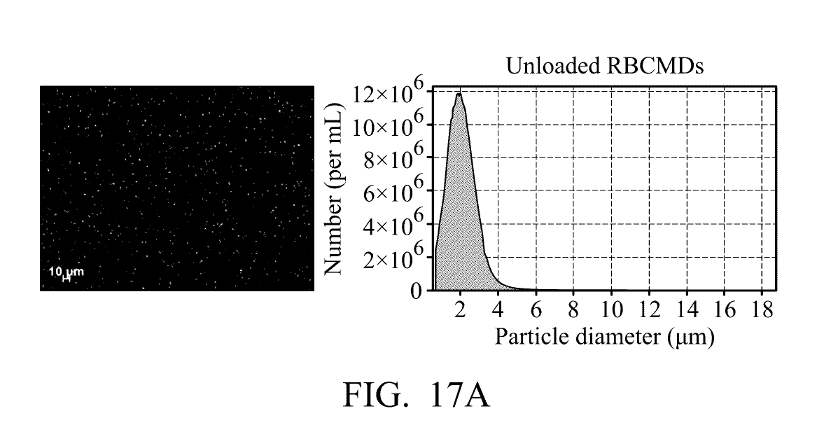

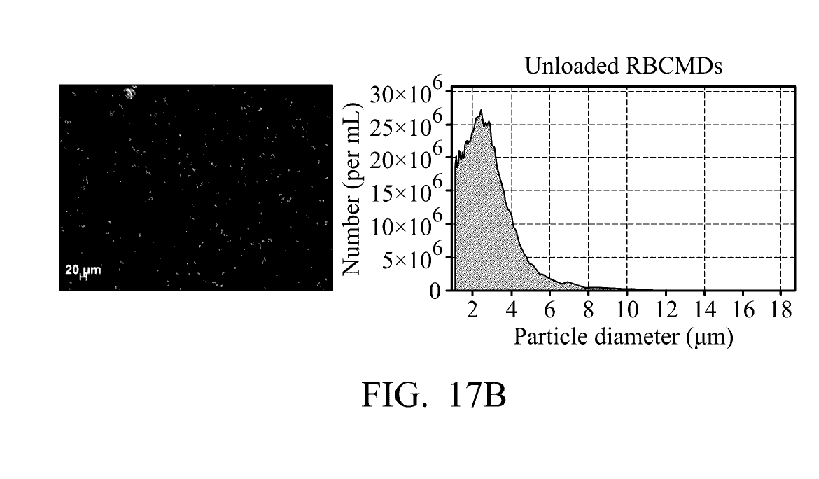

The results of Examples 5 and 6 were shown in FIGS. 17A-17B. It can be found that the resulting droplets have spherical uniform shape, good dispersion, and a particle size of about 2 μm. The particle size of droplets loaded with 100 μg/mL of camptothecin is slightly larger, which is about 2-3 μm.

### Analysis of the Retention of Red Blood Cell Membrane Protein

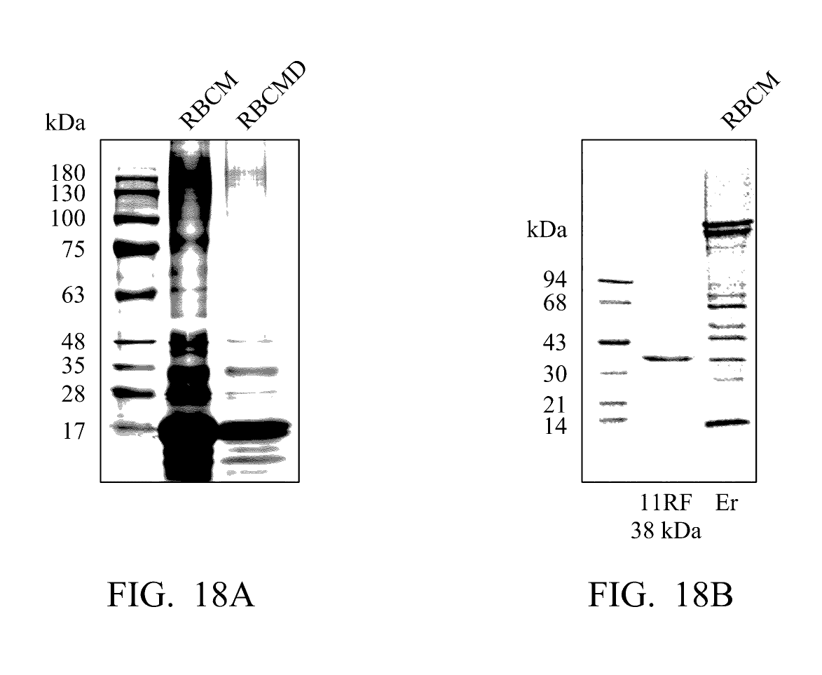

Referring to FIG. 18A, the results show that most of the human red blood cell membrane proteins are still successfully retained on the resulting human red blood cell membrane phase-change droplets after sonication. FIG. 18B shows the composition of human red blood cell membrane proteins published in the reference (Proc. Natl. Acad. Sci. USA Vol. 83, pp. 6975-6979, September 1986), it can be found that the protein composition obtained in the present disclosure is similar to that shown in the reference.

### Analysis of the Drug Loading Efficacy

It was found that the LE % of the phase-change droplets loaded with 100 μg/mL of camptothecin is 2.15±0.25%, EE % of the phase-change droplets loaded with 100 μg/mL of camptothecin is 97.41±15.70%. The LE % of the phase-change droplets loaded with 200 μg/mL of camptothecin is 3.11±0.05%, EE % of the phase-change droplets loaded with 200 μg/mL of camptothecin is 81.38±1.31%. The LE % of the phase-change droplets loaded with 400 μg/mL of camptothecin is 3.13±0.26%, EE % of the phase-change droplets loaded with 400 μg/mL of camptothecin is 62.19±6.67%. It reveals that with the rise of the initial drug concentration, LE % gradually increases (2.15% to 3.13%), while EE % gradually decreases (97.41% to 62.19%).

Therefore, according to these data, appropriate drug concentration can be selected to produce phase-change droplets with high efficacy of drugs use (100 μg/mL, EE %=97.41%) or phase-change droplets with high drug loading (200 μg/mL, LE %=3.11%). The following experiments will use the phase-change droplets loaded with 100 μg/mL and 200 μg/mL of camptothecin to conduct test.

### High-Speed Image Observation of the Ultrasound Triggered Droplet Vaporization

As shown in FIG. 19, by the images captured by high-speed photographing, it can be found that before a single ultrasonic irradiation (before ADV), the human red blood cell membrane phase-change droplets appears to be spherical with small particle size. After ultrasonic irradiation (after ADV), lots of bubbles with particle sizes 5 times larger were produced (larger bubbles may be observed since the different optical focal planes), which is conformed with the theoretical size of vaporization from droplets to bubbles. It reveals that the resulting human red blood cell membrane phase-change droplets can be vaporized by single ultrasound trigger to form larger gas microbubbles having property of ultrasound triggered phase-change droplets. Also, it is confirmed that the resulting human red blood cell membrane phase-change droplets are successfully loaded with perfluoropentane.

### Evaluation of Damage to Cancer Cells of the Ultrasound Triggered Drug Release

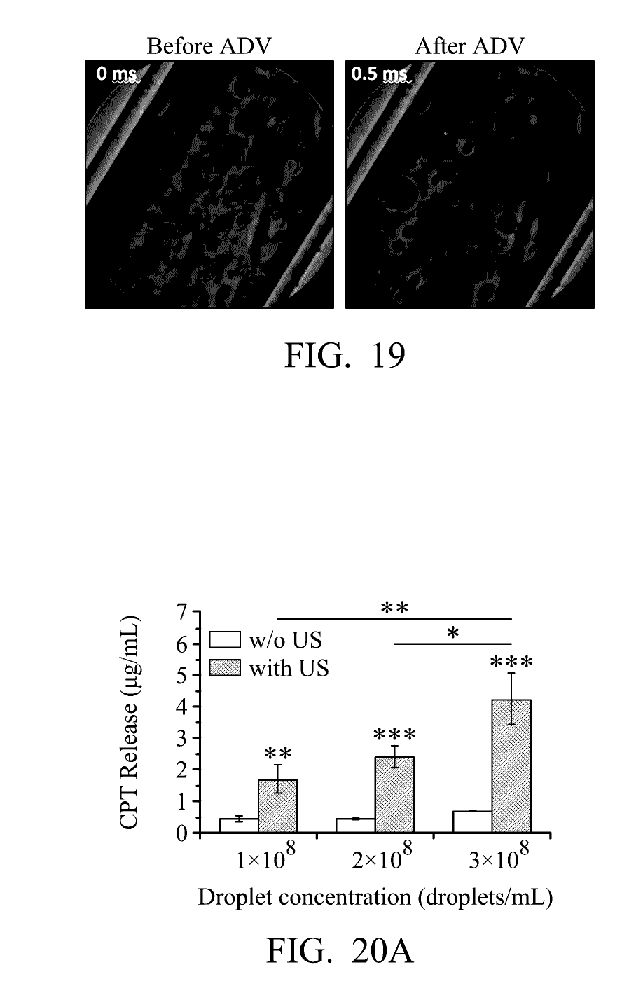

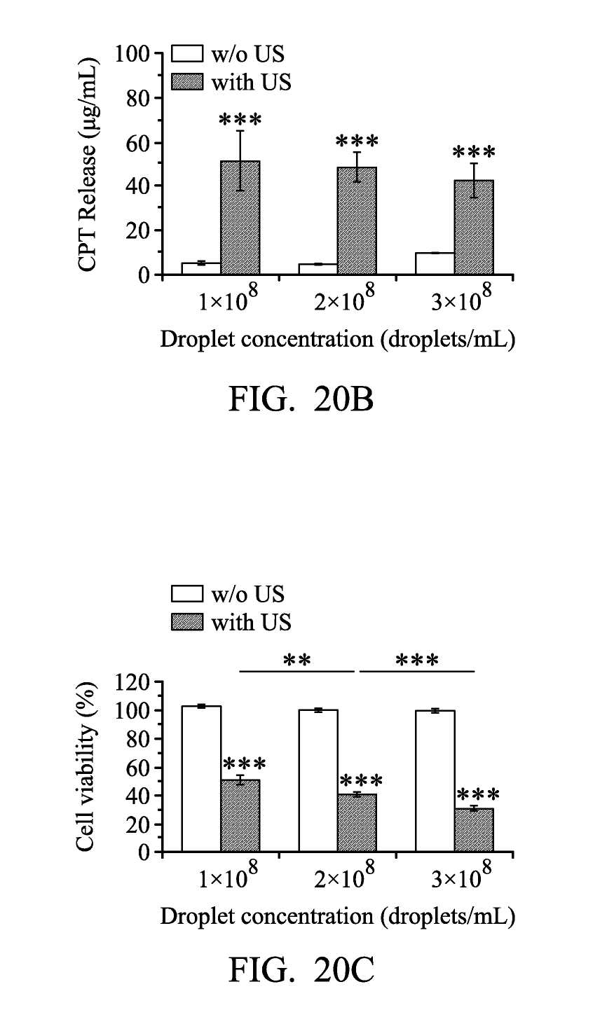

After different amounts of human red blood cell membrane phase-change droplets loaded with camptothecin were irradiated with HIFU three minutes, concentration of released camptothecin was analyzed to evaluate the ultrasound triggered drug release efficacy. FIG. 20A illustrates that the significant differences of drug release concentrations between different dosages of red blood cell membrane phase-change droplets before and after HIFU irradiation. Under the concentration of 1×10⁸, 2×10⁸, and 3×10⁸ droplets/mL whole blood, the drug release concentration of non-irradiated groups are 0.45±0.09 μg/mL, 0.41±0.04 μg/mL and 0.68±0.02 μg/mL, respectively, whereas the drug release concentration of irradiated groups are 1.71±0.46 μg/mL, 2.42±0.33 μg/mL and 4.25±0.81 μg/mL, respectively. Ultrasound triggered drug release concentration and the dosage of droplets have positive correlation.

FIG. 20B illustrates the drug release efficacy of different droplets dosages after HIFU irradiation. The drug release efficacy of non-irradiated groups are all below 7%, whereas the drug release efficacy of irradiated group is about 51% under the concentration of 1×10⁸ droplets/mL whole blood, about 48% under the concentration of 2×10⁸, and slightly decreases to about 42% under the concentration of 3×10⁸. The phenomenon of lower drug release efficacy under higher concentration is similar to that of murine red blood cell membrane phase-change droplets, the reason may be that since droplets with higher concentration causes more ultrasonic reflection and scattering, thereby reduce the drug release efficacy by a shadowing effect formed in the internal droplets.

In order to verify whether the drug release triggered by ultrasound still has an anticancer effect, ultrasound triggered drug release sample was diluted 20-fold and co-cultured with cancer cells (Hela cells) for 24 hours, then a cell viability assay was conducted. Results are shown in FIG. 20C. Non-irradiated group does not have a significant effect on cancer cell viability (viability remains greater than 99%), whereas in the irradiated group, the cancer cell viability are respectively 51%, 40%, and 31% under concentration of 1×10⁸, 2×10⁸ and 3×10⁸ droplets/mL whole blood. It reveals that the camptothecin released by ultrasound trigger still has significant anticancer effect.

The above results confirm that human red blood cell membrane phase-change droplets can be triggered by ultrasound to promote drug release, and can be used as drug carriers which can be remotely triggered, and the drug release triggered by ultrasound still has an anticancer effect. Compared to murine red blood cell membrane phase-change droplets (RBCMD), the triggered drug release efficacy of human red blood cell membrane phase-change droplets under HIFU irradiation is slight higher than that of murine red blood cell membrane phase-change droplets. It may be related to both of the cell membrane compositions. However, the detailed mechanism remains to be determined.

### In Vitro B-Mode Ultrasound Development

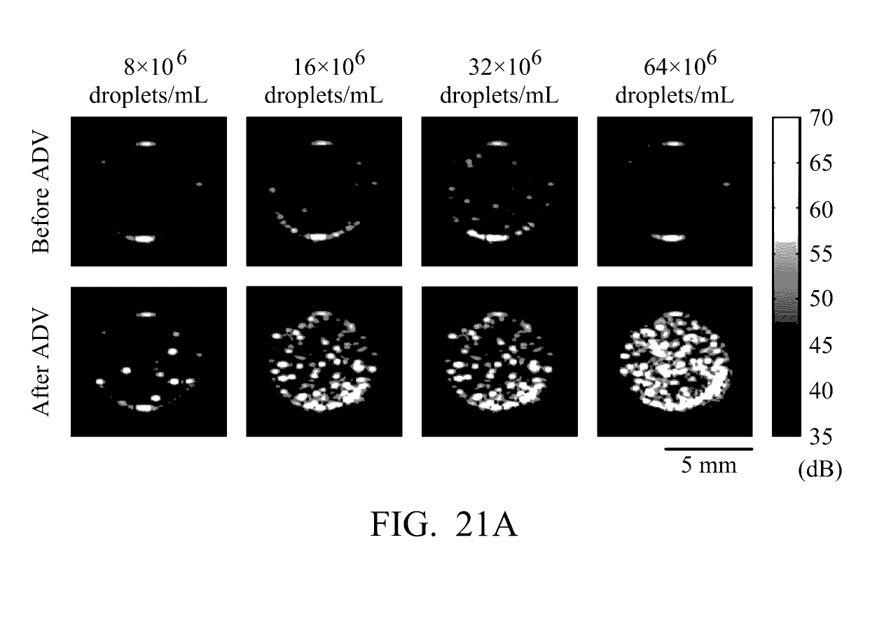

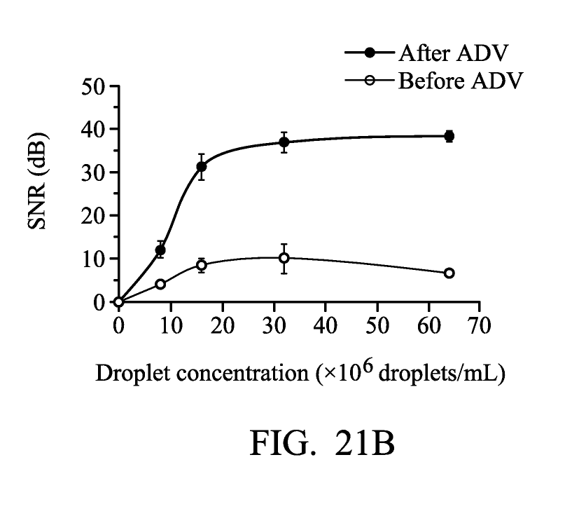

FIG. 21A illustrates the B-mode ultrasound echo images of the red blood cell membrane phase-change droplets in different concentrations before and after the acoustic droplet vaporization (ADV). It can be observed in the images that there are significant differences in B-mode image contrast before and after the ultrasound triggered vaporization. After vaporization, bubbles were produced and thereby the ultrasound reflective signals were enhanced and resulted in bright white images.

FIG. 21B illustrates diagram made of the quantified result of signal to noise ratio (SNR) of the ultrasound reflective signals. As the concentration of red blood cell phase-change droplets increases, the produced signal to noise ratio (SNR) of B-mode also gradually increases, the highest value can reach 38.4±1.3 dB. However, a saturation value was approached under a concentration of 32×10⁶ droplets/mL whole blood

The B-mode images before and after the vaporization triggered by ultrasound reveal that human red blood cell membrane phase-change droplets also have the ability and potential to be used as an ultrasound contrast agent. Compared with the murine red blood cell membrane phase-change droplets, the signal differences before and after the triggered droplets vaporization of the human red blood cell membrane phase-change droplets is more obvious than that of murine red blood cell membrane phase-change droplets. The signal intensity produced by human red blood cell membrane phase-change droplets is also stronger for about 10 dB. Therefore, the human red blood cell membrane phase-change droplets may have better application potential.

### Evaluation of Physical Damage to Cancer Cells of the Ultrasound Triggered Droplet Vaporization

In order to quantify and verify the damage towards adjacent cancer cells by the physical power generated by larger level droplet vaporization, the lymphoma cells (BJAB cells) were mixed with human red blood cell membrane phase-change droplets and added to gel ultrasound phantoms, then irradiated by HIFU to trigger vaporization. Vaporized cell mixed solution was collected and centrifuged to remove fragments.

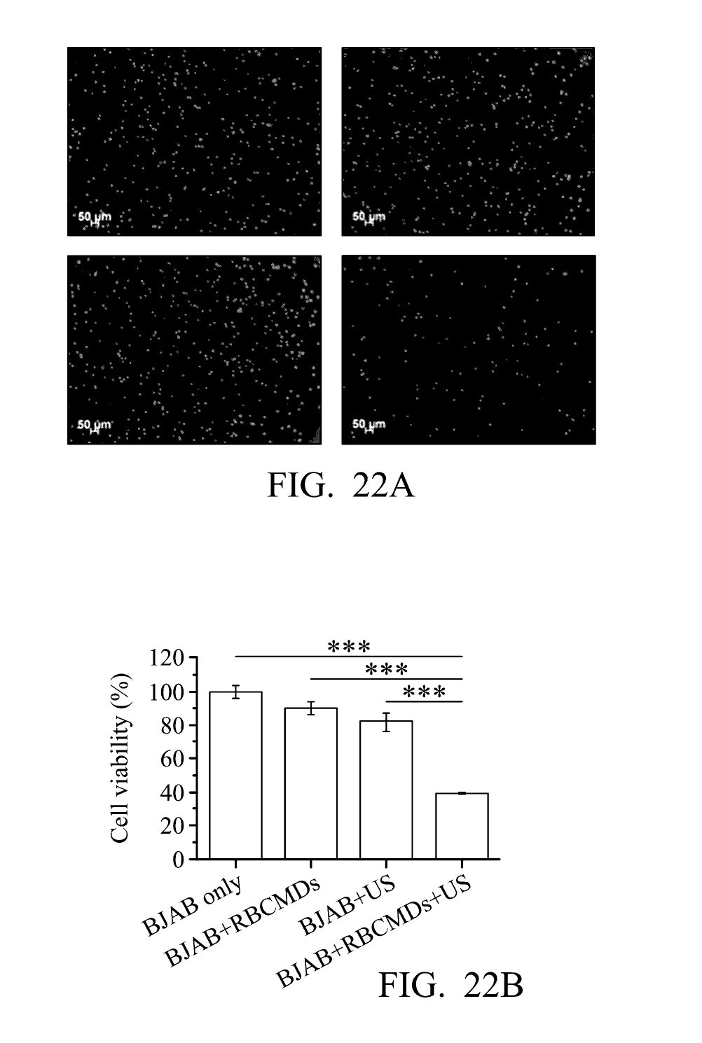

It is observed via microscope that the number of cancer cells (BJAB+RBCMDs+US) significantly decreased in the group with human red blood cell membrane phase-change droplets. Conversely, control groups of pure cancer cells (BJAB), cancer cells with human red blood cell membrane phase-change droplets (BJAB+RBCMD), and cancer cells with ultrasonic irradiation (BJAB+US) do not have significant differences in cell number, as shown in FIG. 22. After conducting cell survival assay, it can be found that the viability of control group (non-irradiated) are still more than 85%, whereas the cell viability of groups with red blood cell membrane phase-change droplets and irradiated by ultrasound decreases to 38%, as shown in FIG. 22B. It reveals that red blood cell membrane phase-change droplets accompanied by the blasting power generated by ultrasound triggered vaporization can cause physical destroy to the adjacent target cells such as cancer cells and result in reduction of viability, thereby has application potential on cancer treatment and destruction of partial tumor blood vessel wall.

The present disclosure successfully prepares phase-change droplets made of self-derived biomembrane which can be used as ultrasound contrast agent, and also successfully prepares a drug carrier which is able of being triggered by ultrasound to release drug, and explores its applications on the medical diagnosis and treatment.

Furthermore, the biomembrane phase-change droplets provided by the present disclosure can further include fluorocarbon nanoparticles such as fluorocarbon iron oxide nanoparticles, fluorocarbon gold nanoparticles, etc. For the drug carriers prepared from the above biomembrane phase-change droplets, the magnetism of the fluorocarbon iron oxide nanoparticles can be used to guide the drug carriers to a specific position to conduct magnetic heat treatment. Alternatively, IR irradiation can be applied to the drug carrier loaded with fluorocarbon gold nanoparticles to trigger the drug carriers to release drug or conduct a photothermal therapy.

Biomembrane phase-change droplets provided by the present disclosure have uniform size with a diameter of about 1.7-2 μm and good dispersion and membrane protein retention. Compared to artificial synthesized phase-change droplets, biomembrane phase-change droplets provided by the present disclosure have good biocompatibility and physiological stability, effectively reduce the attack of immune system, significant reduction of macrophage uptake. Moreover, bubbles generated by ultrasonic irradiation trigger can be used as ultrasound contrast agent and further enhance the ultrasound development. On the other hand, the drug carriers provided by the present disclosure also have uniform size with a diameter of about 1.7-3 μm, and a drug loading of about 0.8-4 wt % (anticancer drug camptothecin; CPT). In addition to the aforementioned characteristics and advantages, biomembrane phase-change droplets of the present disclosure can further release drug by ultrasonic remote control, thereby drugs can be delivered at a specific time and position.

Ultrasound-irradiated drug carriers can promote about 40-50% of drug release and about 50-70% of cancer cell death. Bubbles generated by ultrasound triggered vaporization can significantly enhance the echo signal of ultrasound for about 30 dB, and physically damage adjacent cancer cells by blasting power to cause about 50-60% of cancer cell death. In animal tests, in vivo vaporization of biomembrane phase-change droplets can also be triggered by ultrasound to enhance the ultrasound development, and physically damage adjacent cells by blasting power.

The present disclosure demonstrates that such biomembrane phase-change droplets or drug carriers which are capable of being remotely triggered by ultrasound can effectively cause physical damage to cancer cells in vitro and in vivo. In addition, the released drugs of the drug carriers still retain the effect of inhibition of cancer cell survival. Therefore, biomembrane phase-change droplets and drug carriers have a very high value in clinical treatment and will be used as a medicine for the cancer therapy.

While the disclosure has been described by way of example and in terms of the embodiments, it is to be understood that the disclosure is not limited to the disclosed embodiments. It will be apparent to those skilled in the art that various modifications and variations can be made to the disclosed embodiments. Therefore, a true scope of the disclosure being indicated by the following claims and their equivalents.

What is claimed is:

1. A biomembrane phase-change droplet (PCD), comprising:
a hydrophobic liquid core; and
a phospholipid-containing biomembrane encapsulating the hydrophobic liquid core;
wherein the hydrophobic liquid core is vaporized by ultrasonic irradiation.

2. The biomembrane phase-change droplet (PCD) as claimed in claim 1, wherein the weight ratio of the hydrophobic liquid core and the biomembrane is 1-20: 20-1.

3. The biomembrane phase-change droplet (PCD) as claimed in claim 1, wherein the particle size distribution of the biomembrane phase-change droplet (PCD) is between 0.1-5 micrometer.

4. The biomembrane phase-change droplet (PCD) as claimed in claim 1, wherein the hydrophobic liquid core comprises a fluorocarbons, other hydrophobic solvents, or a combination thereof.

5. The biomembrane phase-change droplet (PCD) as claimed in claim 4, wherein the fluorocarbons comprises C₃F₈, C₄F₁₀, C₅F₁₂, C₆F₁₄, or a combination thereof.

6. The biomembrane phase-change droplet (PCD) as claimed in claim 1, wherein the biomembrane comprises red blood cell membrane, stem cell membrane, or another cell membrane having phospholipid bilayer structure.

7. The biomembrane phase-change droplet (PCD) as claimed in claim 1, wherein the biomembrane is derived from mammalian cell membrane.

8. The biomembrane phase-change droplet (PCD) as claimed in claim 1, wherein the ultrasound comprises a high-intensity focused ultrasound (HIFU).

9. The biomembrane phase-change droplet (PCD) as claimed in claim 1, which further comprises a fluorocarbon nanoparticle, dispersing in the hydrophobic liquid core, wherein the weight percentage of the fluorocarbon nanoparticle is 0.1-5 wt %, based on the weight of the hydrophobic liquid core.

10. The biomembrane phase-change droplet (PCD) as claimed in claim 9, wherein the fluorocarbon nanoparticle comprises fluorocarbon iron oxide nanoparticles, fluorocarbon gold nanoparticles, fluorocarbon silicon oxide nanoparticles, or a combination thereof.

11. Use of the biomembrane phase-change droplet (PCD) as claimed in claim 1 as an ultrasound contrast agent.

12. A drug carrier, comprising:
a biomembrane phase-change droplet (PCD) as claimed in claim 1; and
a hydrophobic drug, embedded on the biomembrane of the biomembrane phase-change droplet (PCD) as claimed in claim 1,
wherein the hydrophobic drug is present in an amount of 1-10 wt %, based on the weight of the drug carrier.

13. The drug carrier as claimed in claim 12, wherein the hydrophobic drug comprises camptothecin (CPT), paclitaxel, chlorin e6 (Ce6), or a combination thereof.

14. The drug carrier as claimed in claim 12, wherein the hydrophobic drug is released as the vaporization of the hydrophobic liquid core.

15. Use of the drug carrier as claimed in claim 12 as an ultrasound contrast agent.

16. Use of the drug carrier as claimed in claim 12 to manufacture a medicine for cancer therapy.
